(19) United States
(12) Patent Application Publication
(10) Pub. No.: US 2002/0133844 A1
(43) Pub. Date: Sep. 19, 2002

(54) Regulation of Th2 cell activity by modulation of NFATp and NFAT4 activity

(75) Inventors: Laurie H. Glimcher, West Newton, MA (US); Ann M. Ranger, Brighton, MA (US); Mohamed Oukka, Brighton, MA (US)

Correspondence Address: LAHIVE & COCKFIELD, 28 STATE STREET, BOSTON, MA, 02109, US

(73) Assignee: President and Fellows of Harvard College

(21) Appl. No.: 10/035,688

(22) Filed: Nov. 8, 2001

Related U.S. Application Data
(63) Continuation of application No. 09/181,716, filed on Oct. 28, 1998, now abandoned.

(51) Int. Cl. A01K 67/027; C12Q 1/00; G01N 33/53; G01N 33/567

(52) U.S. Cl. 800/18; 435/4; 435/7.2

(57) ABSTRACT

The invention demonstrates that NFATp and NFAT4 are required for the control of lymphocyte homeostasis and act as selective repressors of Th2 cells. The invention provides mice deficient in both NFATp and NFAT4 that exhibit a phenotype characteristic of increased Th2 cell activity. Methods for identifying modulators of Th2 cell activity, using either cells deficient in both NFATp and NFAT4, mice deficient in both NFATp and NFAT4, or indicator compositions containing both NFATp and NFAT4, are provided. Methods of regulating Th2 cell activity using agents that modulate the activity of NFATp and NFAT4 are also provided. Methods for diagnosing disorders associated with aberrant Th2 cell activity by assessing changes in NFATp and/or NFAT4 expression, are also provided.

### GOVERNMENT FUNDING

Work described herein was supported, at least in part, under grant AI/AG37833 awarded by the National Institutes of Health. The U.S. government therefore may have certain rights in this invention.

### BACKGROUND OF THE INVENTION

Nuclear Factor of Activated T cells (NFAT) is a critical regulator of early gene transcription in response to TCR mediated signals. First identified as a transcriptional regulatory complex important for the expression of the T cell cytokine, IL-2 [Rao, A. et al., Annu. Rev. Immunol. 15:707 (1997); Shaw, J. et al., Science 241:202 (1988); Crabtree, G., Science 249:355 (1989)] NFAT target sequences have since been identified in the promoters of multiple cytokine genes, including IL-4, GM-CSF, IL-3 and TNFα [Miyatake, S. et al., Mol. Cell. Biol. 11:5894 (1991); Goldfeld, A. E., et al., J. Exp. Med. 178:1365 (1993); Masuda, E. S. et al., Mol. Cell. Biol. 13:7399 (1993); DelPrete, G. F. et al., J. Clin. Invest. 88:346 (1991); Rooney, J. W. et al., EMBO J. 13:625 (1994); Chuvpilo, S. et al., Nuc. Acid Res. 21:5694 (1993); Cockerill, P. N. et al., Mol. Cell. Biol. 15:2071 (1995); Rooney, J. W. et al., Immunity 2:545 (1995)]. NFAT target sequences have also been identified in the promoters of the FasL and CD40L cell surface receptors [Tsitsikov, E. N. et al., Immunology 31:895 (1994); Latinis, K. M. et al., J. Immunol. 158:4602 (1997)]. NFAT expression has also been observed in B lymphocytes [Venkataraman L. et al., Immunity 1:189 (1994); Choi, M. S. K. et al., Immunogenetics 1: 189 (1994)] as well as in multiple cell types [Timmerman, L. A. et al., J. Immunol. 159:2735 (1997)] within the innate immune system (NK, macrophage, mast cells) although the endogenous target genes regulated by NFAT in these cells have not yet been identified. More recently. NFATc has been shown to regulate HIV-1 replication in T cells [Kinoshita, S. et al., Immunity 6:235 (1997)].

The NFAT complex contains a cytoplasmic subunit and a ras/protein kinase C-responsive inducible nuclear component [Flanagan, W. M. et al., Nature 352:803 (1991)] composed in part of AP-1 family member proteins [Rooney, J. W. et al., Immunity 2:545 (1995); Jain, J. et al., Nature 356:801 (1992); Jain, J. et al., Nature 365:352 (1993); Rooney, J. et al., Moll. Cell. Biol. 15:6299 (1995); Boise, L. H. et al., Mol. Cell. Biol. 13:1911 (1993)]. Following activation through the T cell receptor (TCR), BCR or CD40 accessory molecules, the cytoplasmic subunit translocates into the nucleus. NFAT nuclear translocation is controlled by the calcium-regulated phosphatase calcineruin which is a target of the immunosuppressive drugs cyclosporin A (CsA) and FK506 [Flanagan, W. M. et al., Immunity 6:235 (1997); Beals, C. R. et al., Genes Dev. 11:824 (1997); Clipstone, N. A. et al., Nature 357:695 (1992)]. Treatment of T cells with CsA or FK506 prevents NFAT nuclear translocation and subsequent activation of cytokine gene transcription [Emmel, E. A. et al., Science 246:1617 (1989)].

There are currently four NFAT genes encoding the cytoplasmic subunit, NFATp (NFATc2, NFAT1), NFATc (NFATc1, NFAT2), NFAT3 (NFATc4). NFAT4 (NFATc3 NFATx) [Northrop, J. P. et al., Nature 369:497 (1994); McCaffrey, P. G. et al., Science 262:750 (1993); Hoey, T. et al., Immunity 2:461 (1995); Masuda, E. S. et al., Mol Cell. Biol. 15:2697 (1995); Ho, S. N. et al., J. Biol. Chem. 270:19898 (1995)]. In vitro, all these factors can bind to and transactivate the promoters of multiple cytokine genes, although in T cell extracts only NFATc and NFATp bind to these sites [Timmerman, L. A. et al., J. Immunol. 159:2735 (1997)]. The sequence variability among NFAT family members in N- and C-terminal regions that contain transactivation domains [Luo, C. et al., J. Exp. Med. 184:141 (1996)], together with their differing tissue distribution [Masuda, E. S. et al., Mol. Cell. Biol. 15:2697 (1995)] suggested functional differences among NFAT family members.

### SUMMARY OF THE INVENTION

This invention pertain to methods and compositions relating to regulation of Th2 cell activity (e.g., Th2 cytokine production) by modulation of both NFATp and NFAT4 activity. It has now been discovered that NFATp and NFAT4 are required for the control of lymphocyte homeostasis and act as selective repressors of Th2 cells. The invention is based, at least in part, on the observation that mice lacking both NFATp and NFAT4 exhibit features characteristic of profound increases in Th2 cell activity, including allergic blepharitis, interstitial pneumonitis and granuloma formation, with a dramatic and selective increase in Th2 cytokine production and a corresponding 10³ to 10⁴ fold increase in serum IgG1 and IgE levels. Mice lacking both NFATp and NFAT4 also develop a profound lymphoproliferative disorder characterized by the accumulation of peripheral T and B cells with a memory/activated phenotype, likely due to defective FasL expression. Thus, the combined inhibition of NFATp and NFAT4 results in greatly stimulated Th2 cell activity and accordingly, Th2 cell activity can be regulated by modulating the activity of NFATp and NFAT4.

One aspect of the invention pertains to a mouse comprising in its genome a first exogenous DNA molecule that functionally disrupts a NFATp gene of said mouse and a second exogenous DNA molecule that functionally disrupts a NFAT4 gene of said mouse, wherein said mouse exhibits a phenotype characterized by increased Th2 cytokine production, relative to a wildtype mouse. In a preferred embodiment, the phenotype of the mouse is further characterized by: (a) blepharatis; (b) interstitial pneumonitis; (c) splenomegaly and lymphadenopathy; and (d) increased levels of serum IgG1 and IgE, relative to a wildytype mouse.

In view of the readily detectable phenotype of mice lacking NFATp and NFAT4 provided by the invention, these mice and lymphoid cells thereof are particularly useful in methods for identifying modulators of Th2 cytokine production. Accordingly, another aspect of the invention pertains to a method of identifying a compound that regulates Th2 cell activity The method involves:

a) contacting lymphoid cells deficient in NFATp and NFAT4 with a test compound; and

b) determining the effect of the test compound on an indicator of Th2 cell activity of the lymphoid cells. The test compound is identified as a regulator of Th2 cell activity based on the ability of the test compound to modulate an indicator of Th2 cell activity of the lymphoid cells deficient in NFATp and NFAT4. In one embodiment, the lymphoid cells deficient in NFATp and NFAT4 are in a mouse that is deficient in NFATp and NFAT4 and the lymphoid cells are contacted with the test compound by administering the test compound to the mouse. In another embodiment, the lymphoid cells deficient in NFATp and NFAT4 are isolated from a mouse deficient in NFATp and NFAT4 and the lymphoid cells are contacted with the test compound by culturing the test compound with the isolated lymphoid cells deficient in NFATp and NFAT4. In a preferred embodiment, a compound identified by the method inhibits Th2 cytokine production (i.e., counteracts the increased Th2 cytokine production that is exhibited by the lymphoid cells deficient in NFATp and NFAT4).

In view of the demonstation herein that NFATp and NFAT4 function together as repressors of Th2 cell activity, compositions containing these two factors can be used in methods to identify modulators of Th2 cell activity. Accordingly, another aspect of the invention pertains to a method of identifying a compound that modulates Th2 cell activity, comprising

a) providing at least one indicator composition comprising NFATp protein and NFAT4 protein;

b) contacting the at least one indicator composition with each member of a library of test compounds;

c) selecting from the library of test compounds a compound of interest that modulates the activity of NFATp protein and NFAT4 protein; and

d) determining the effect of the compound of interest on Th2 cell activity to thereby identify a compound that modulates Th2 cell activity.

In one embodiment, the indicator composition comprises cells that expresses NFATp protein and/or NFAT4 protein, for example a cell that has been engineered to express the NFATp protein and another cell that has been engineered to express the NFAT4 protein, by introducing into the cells an expression vector encoding either the NFATp protein or the NFAT4 protein. In another embodiment, the indicator composition is a cell free composition. In yet another embodiment, the indicator composition is at least one cell that expresses an NFATp protein. an NFAT4 protein and at least one target molecule, and the ability of the test compound to modulate the interaction of the NFATp protein and the NFAT4 protein with the at least one target molecule is monitored. In still another embodiment, the indicator composition comprises at least one indicator cell, wherein the indicator cell(s) comprise an NFATp protein, an NFAT4 protein and at least one reporter gene responsive to the NFATp protein and/or the NFAT4 protein.

Yet another aspect of the invention pertains to methods for modulating Th2 cell activity by contacting lymphoid cells with a modulator of NFATp and NFAT4 activity such that Th2 cell activity within the lymphoid cells is modulated. In one embodiment, the modulator inhibits NFATp and NFAT4 activity. In another embodiment, the modulator stimulates NFATp and NFAT4 activity.

Still another aspect of the invention pertains to a method of diagnosing a subject for a disorder associated with aberrant Th2 cell activity by detecting a change(s) in the expression of NFATp and/or NFAT4 in cells of the subject. For example, the invention provides a method comprising:

(a) detecting expression of NFATp and NFAT4 in lymphoid cells of a subject suspected of having a disorder associated with aberrant Th2 cell activity;

(b) comparing expression of NFATp and NFAT4 in lymphoid cells of said subject to a control that is not associated with aberrant Th2 cell activity; and

(c) diagnosing the subject for a disorder based a change in expression of NFATp or NFAT4 in lymphoid cells of the subject as compared to the control.

### BRIEF DESCRIPTION OF THE DRAWINGS

FIGS. 1A-C are bar graphs demonstrating extremely elevated levels of Th2 cytokines in NFATp/NFAT4 doubly-deficient (−/− or DKO) mice as compared to wildtype (+/+ or WT) mice. FIG. 1A shows increased Th2 cytokine production and FIG. 2B shows decreased Th1 cytokine production upon primary stimulation of DKO spleen cells. Numbers above the bars represent the approximate DKO/WT ratio for each cytokine. FIG. 2C shows secondary stimulation of spleen cells from DKO mice. Note that IL-4 is expressed on a log scale.

### DETAILED DESCRIPTION OF THE INVENTION

This invention pertains to methods and compositions relating to modulation of Th2 cell activity by modulation of NFATp and NFAT4 activity. The invention is based, at least in part, on the surprising discovery that mice deficient in both NFATp and NFAT4 exhibit features characteristic of profound increases in Th2 cell activity, including allergic blepharitis, interstitial pneumonitis and granuloma formation, with a dramatic and selective increase in Th2 cytokine production and a corresponding 103 to 104 fold increase in serum IgG1 and IgE levels. Mice lacking both NFATp and NFAT4 also develop a profound lymphoproliferative disorder characterized by the accumulation of peripheral T and B cells with a memory/activated phenotype, likely due to defective FasL expression. The results described herein demonstrate that NFATp and NFAT4 are required for the control of lymphocyte homeostasis and act as selective repressors of Th2 cells. Accordingly, Th2 cell activity can be regulated by modulating the activity of NFATp and NFAT4.

One aspect of the invention pertains to a mouse that is deficient in NFATp and NFAT 4 expression. Another aspect of the invention pertains to use of these mice, or cells from these mice. to identify modulators of Th2 cell activity. For example, in one aspect, the invention pertains to a method of identifying a compound that regulates Th2 cell activity in which lymphoid cells deficient in NFATp and NFAT4 are contacted with a test compound to identify compounds that regulates Th2 cell activity (e.g., that inhibit Th2 cell activity). In another embodiment of these screening assays, an indicator composition that includes NFATp and NFAT4 is used to identify and select compounds that modulate the activity of these factors and then the effect of the selected compounds on Th2 cell activity is evaluated.

In another aspect, the invention pertains to method for regulating Th2 cell activity, either in vitro or in vivo, using modulators of NFATp and NFAT4 activity. In one embodiment, lymphoid cells (e.g., lymphoid cells isolated from a subject) are contacted with a modulator compound by culturing the lymphoid cells with the modulator in vitro. The lymphoid cells, or mature Th2 cells that have formed upon proliferation and differentiation of the lymphoid cells in culture, can then be readministered to a subject. In another embodiment, aberrant Th2 cell activity in a subject is modulated by administering to the subject a therapeutically effective amount of a modulator of NFATp and NFAT4 activity such that aberrant Th2 cell activity in a subject is modulated. Use of modulators that inhibit or stimulate NFATp and NFAT4 activity are encompassed by these modulatory methods of the invention.

In yet another aspect, the invention pertains to a method of diagnosing a subject for a disorder associated with aberrant Th2 cell activity by detecting a change in expression of NFATp and/or NFAT4 in lymphoid cells of a subject suspected of having a disorder associated with aberrant Th2 cell activity.

So that the invention may be more readily understood, certain terms are first defined.

As used herein, the term “NFATp” is intended to refer to a protein, also known in the art as NFAT1, that is a DNA binding protein, expressed in T cells, and has an amino acid sequence as described in, for example, U.S. Pat. No. 5,656,452 by Rao et al., U.S. Pat. No. 5,612,455 by Hoey, or other mammalian homologs thereof.

As used herein, the term “NFAT4” is intended to refer to a protein that is a DNA binding protein, expressed preferentially in thymocytes, and has an amino acid sequence as described in Masuda, E. S. et al. (1995) Mol. Cell. Biol. 15:2697-2706 and Genbank Accession No. U85430, or other mammalian homologs thereof.

As used herein, the term “Th2 cell activity” refers to activity of a subpopulation of CD4⁺ T cells that is characterized by the production of one or more cytokines selected from IL-4, IL-5, IL-6, IL-10 and IL-13, and that is associated with efficient B cell “help” provided by the Th2 cells (e.g., enhanced IgG1 and/or IgE production). Th2 cell activity can be assessed by monitoring an indicator of Th2 cell activity, such as levels of Th2-associated cytokine production, levels of serum IgG1 and/or IgE or infammations that result from upregulated Th2 cell activity, such as blepharitis, interstitial pneumonitis and/or increased mast cell numbers and granumolas in spleen and lymph node.

As used herein, the term “Th2-associated cytokine” is intended to refer to a cytokine that is produced preferentially or exclusively by Th2 cells rather than by Th1 cells. Examples of Th2-associated cytokines include IL-4, IL-5, IL-6, IL-10 and IL-13.

As used herein, the various forms of the terms “modulate” or “regulate” are intended to include stimulation (e.g., increasing or upregulating a particular response or activity) and inhibition (e.g., decreasing or downregulating a particular response or activity).

As used herein, the term “contacting” (i.e., contacting a cell e.g. a lymphoid cell, with an compound) is intended to include incubating the compound and the cell together in vitro (e.g., adding the compound to cells in culture) and administering the compound to a subject such that the compound and cells of the subject are contacted in vivo. The term “contacting” is not intended to include exposure of lymphoid cells to NFATp/NFAT4 modulators that may occur naturally in a subject (i.e., exposure that may occur as a result of a natural physiological process).

As used herein, the term “test compound” is intended to refer to a compound that has not previously been identified as, or recognized to be, a modulator of NFATp and/or NFAT4 activity and/or of Th2 cell activity.

The term “library of test compounds” is intended to refer to a panel comprising a multiplicity of test compounds.

As used herein, the term “cells deficient in NFATp and NFAT4” is intended to include cells of a subject that are naturally deficient in NFATp and NFAT4, as wells as cells of an NFATp/NFAT4 deficient mouse that have been altered such that they are deficient in NFATp and NFAT4. The term “cells deficient in NFATp and NFAT4” is also intended to include cells isolated from an NFATp/NFAT4 deficient mouse or a subject that are cultured in vitro.

As used herein, the term “NFATp and NFAT4 deficient mouse” refers to a mouse in which the endogenous NFATp and NFAT4 genes have been altered by homologous recombination between the endogenous genes and exogenous DNA molecules introduced into a cell of the animal, e.g., an embryonic cell of the mouse, prior to development of the mouse, such that the endogenous NFATp and NFAT4 genes are altered, thereby leading to either no production of NFATp/NFAT4 or production of mutant forms of NFATp/NFAT4 having deficient NFATp/NFAT4 activity. Preferably, the activity of NFATp and NFAT4 is entirely blocked, although partial inhibition of NFATp and NFAT4 activity in the mouse is also encompassed. Preferably, the NFATp/NFAT4 doubly deficient mouse is made by cross-breeding a mouse deficient in NFATp with a mouse deficient in NFAT4 and selecting for progeny that are doubly deficient in NFATp and NFAT4.

As used herein, the term “indicator composition” refers to a composition that includes NFATp and NFAT4 proteins, for example, a cell that naturally expresses NFATp and NFAT4 proteins, a cell that has been engineered to express the NFATp and NFAT4 proteins by introducing an expression vector(s) encoding the NFATp and NFAT4 proteins into the cell, or a cell free composition that contains NFATp and NFAT4 (e.g, naturally-occurring NFATp and NFAT4 or recombinantly-engineered NFATp and NFAT4).

As used herein, the term “engineered” (as in an engineered cell) refers to a cell into which an expression vector(s) encoding the NFATp and/or NFAT4 protein has been introduced.

As used herein, the term “cell free composition” refers to an isolated composition which does not contain intact cells. Examples of cell free compositions include cell extracts and compositions containing isolated proteins.

As used herein, the term “a target molecule” for NFATp and/or NFAT4 refers a molecule with which NFATp and/or NFAT4 can interact, including other proteins and DNA sequences, including for example, the IL-2, IL-4, GM-CSF, TNF-α, IL-3, and IL-4 promoter/enhancer regions, AP-1 protein and IκB protein.

As used herein, the term “reporter gene responsive to NFATp and/or NFAT4” refers to any gene that expresses a detectable gene product, which may be RNA or protein, and whose expression is regulated by NFATp and/or NFAT4. Preferred reporter genes are those that are readily detectable. The reporter gene may also be included in a construct in the form of a fusion gene with a gene that includes desired transcriptional regulatory sequences or exhibits other desirable properties. Examples of reporter genes include, but are not limited to CAT (chloramphenicol acetyl transferase) (Alton and Vapnek (1979), Nature 282: 864-869) luciferase, and other enzyme detection systems, such as beta-galactosidase; firefly luciferase (deWet et al. (1987), Mol. Cell. Biol. 7:725-737); bacterial luciferase (Engebrecht and Silverman (1984), PNAS 1: 4154-4158; Baldwin et al. (1984), Biochemistry 23: 3663-3667); alkaline phosphatase (Toh et al. (1989) Eur. J. Biochem. 182: 231-238, Hall et al. (1983) J. Mol. Appl. Gen. 2: 101), human placental secreted alkaline phosphatase (Cullen and Malim (1992) Methods in Enzymol. 216:362-368) and green fluorescent protein (U.S. Pat. No. 5,491,084; WO 96/23898).

As used herein, the term “NFATp- or NFAT4-responsive element” refers to a DNA sequence that is directly or indirectly regulated by the activity of the NFATp or NFAT4 (whereby activity of NFATp or NFAT4 can be monitored, for example, via transcription of the reporter gene).

As used herein, the term “aberrant” (as in aberrant cTh2 cell activity) refers to Th2 cell activity that deviates from normal Th2 cell activity in a subject. The aberrant Th2 cell activity can either be excessive Th2 cell activity or reduced Th2 cell activity with respect to normal Th2 cell activity in a subject.

As used herein, the term “a modulator of NFATp or NFAT4 activity” is intended to refer to an agent, for example a compound or compounds, which modulates transcription of an NFATp or NFAT4 gene, translation of NFATp or NFAT4 mRNA or activity of an NFATp or NFAT4 protein. Examples of modulators that directly modulate NFATp and/or NFAT4 activity include antisense nucleic acid molecules that bind to NFATp and/or NFAT 4 mRNA or genomic DNA, intracellular antibodies that bind to NFATp and/or NFAT4 intracellularly and modulate (i.e., inhibit) NFATp and/or NFAT4 activity, NFATp and/or NFAT4 peptides that inhibit the interaction of NFATp and/or NFAT4 with a target molecule (e.g., calcineurin) and expression vectors encoding NFATp and/or NFAT4 that allow for increased expression of NFATp and/or NFAT4 activity in a cell, as well as chemical compounds that act to specifically modulate the activity of NFATp and/or NFAT4.

As used herein, an “antisense oligonucleotide” refers to a nucleic acid that comprises a nucleotide sequence which is complementary to a “sense” nucleic acid encoding a protein, e.g., complementary to the coding strand of a double-stranded cDNA molecule, complementary to an mRNA sequence or complementary to the coding strand of a gene. Accordingly, an antisense nucleic acid can hydrogen bond to a sense nucleic acid.

As used herein, the term “intracellular antibody” is intended to include immunoglobulin molecules and immunologically active portions of immunoglobulin molecules, i.e., molecules that contain an antigen binding site which specifically binds (immunoreacts with) an antigen, such as Fab and F(ab′)₂ fragments. The term “intracellular antibody” is also intended to refer to an antibody that functions in an intracellular region of a cell, e.g., the cytoplasm or nucleus, to modulate the expression or activity of the NFATp and/or NFAT4.

As used herein, the term “diagnosing” refers to identifying a disorder in a subject or the susceptibility of a subject to the disorder (e.g., a predisposition to develop a disorder).

Various aspects of the present invention are described in further detail in the following subsections.

1. NFATp/NFAT4 Deficient Mice

One aspect of the invention pertains to a mouse that is deficient in expression of NFATp and NFAT4. The invention provides a mouse comprising in its genome a first exogenous DNA molecule that functionally disrupts a NFATp gene of the mouse and a second exogenous DNA molecule that functionally disrupts a NFAT4 gene of the mouse. The term “exogenous DNA” refers to a DNA molecule that does not naturally occur in that location of the genome of the mouse and that serves to disrupt the natural endogenous gene. The NFATp/NFAT4 mice of the invention exhibit a phenotype characterized by increased Th2 cytokine production, relative to a wildtype mouse. The phenotype of the mice can further characterized by: (a) blepharitis; (b) interstitial pneumonitis; (c) splenomegaly and lymphadenopathy; and (d) increased levels of serum IgG1 and IgE, relative to a wildytype mouse. Other phenotypic features of the mice of the invention are described in detail in the Examples.

NFATp and NFAT4 doubly deficient mice typically are created by homologous recombination. Briefly, to create mice that are deficient in either NFATp or NFAT4 a vector is prepared which contains at least a portion of the NFATp or NFAT4 gene into which a deletion, addition or substitution has been introduced to thereby alter, e.g., functionally disrupt, the endogenous NFATp or NFAT4 gene. For example, a mouse NFATp or NFAT4 gene can be isolated from a mouse genomic DNA library using the mouse NFATp or NFAT4 cDNA as a probe. The mouse NFATp or NFAT4 gene then can be used to construct a homologous recombination vector suitable for altering an endogenous NFATp or NFAT4 gene in the mouse genome. In a preferred embodiment, the vector is designed such that, upon homologous recombination, the endogenous NFATp or NFAT4 gene is functionally disrupted (i.e., no longer encodes a functional protein; also referred to as a “knock out” vector). Alternatively, the vector can be designed such that, upon homologous recombination, the endogenous NFATp or NFAT4 gene is mutated or otherwise altered but still encodes functional protein (e.g., the upstream regulatory region can be altered to thereby alter the expression of the endogenous NFATp or NFAT4 protein).

In the homologous recombination vector, the altered portion of the NFATp or NFAT4 gene is flanked at its 5′ and 3′ ends by additional nucleic acid of the NFATp or NFAT4 gene to allow for homologous recombination to occur between the exogenous NFATp or NFAT4 gene carried by the vector and an endogenous NFATp or NFAT4 gene in an embryonic stem cell. The additional flanking NFATp or NFAT4 nucleic acid is of sufficient length for successful homologous recombination with the endogenous gene. Typically, several kilobases of flanking DNA (both at the 5′ and 3′ ends) are included in the vector (see e.g., Thomas, K. R. and Capecchi, M. R. (1987) Cell 51:503 for a description of homologous recombination vectors). The vector is introduced into an embryonic stem cell line (e.g., by electroporation) and cells in which the introduced NFATp or NFAT4 gene has homologously recombined with the endogenous NFATp or NFAT4 gene are selected (see e.g., Li, E. et al. (1992) Cell 69:915). The selected cells are then injected into a blastocyst of a mouse to form aggregation chimeras (see e.g., Bradley, A. in Teratocarcinomas and Embryonic Stem Cells: A Practical Approach, E. J. Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley, A. (1991) Current Opinion in Biotechnology 2:823-829 and in PCT International Publication Nos.: WO 90/11354 by Le Mouellec et al.; WO 91/01140 by Smithies et al.; WO 92/0968 by Zijlstra et al.; and WO 93/04169 by Berns et al.

NFATp deficient mice created by homologous recombination having a disrupted NFATp gene can be generated for example, as described by Hodge et al. (1996) Immunity 4:397-405, the contents of which are expressly incorporated herein by reference. The targeted exon was in the DNA-binding domain, and its disruption results in the expression of a deleted version of the protein without DNA-binding activity. NFAT4 deficient mice created by homologous recombination having a disrupted NFAT4 gene can be generated, for example, as described by Oukka et al. (1998) Immunity 9:295-304, the contents of which are also expressly incorporated herein by reference. Mice doubly deficient in NFATp and NFAT4 can then be created by cross-breeding the singly deficient mice and selecting for progeny that are deficient in both NFATp and NFAT4, as described in Example 1.

II. Screening Assays To Identify Compounds That Regulate Th2 Cell Activity

A. Assays Using NFATp and NFAT4 Deficient Cells

In one embodiment, the invention provides methods for identifying compounds that modulate Th2 cell activity using cells deficient in NFATp and NFAT4. As described in the Examples, inhibition of NFATp and NFAT4 activity (e.g., by disruption of both the NFATp and NFAT4 genes) leads to greatly increased Th2 cell activity. Accordingly, lymphoid cells from NFATp/NFAT4 doubly deficient mice, having enhanced Th2 cell activity, can be used to identify agents that modulate Th2 cell activity by means other than modulating NFATp or NFAT4 themselves.

In the screening method, lymphoid cells deficient in NFATp and NFAT4 are contacted with a test compound and Th2 activity of the lymphoid cells is monitored. Modulation of Th2 cell activity of the NFATp/NFAT4 deficient lymphoid cells (as compared to an appropriate control such as, for example, untreated cells or cells treated with a control agent) identifies a test compound as a modulator Th2 cell activity. In one embodiment, the test compound is administered directly to an NFATp/NFAT4 deficient mouse to identify a test compound that modulates in vivo Th2 cell activity. In another embodiment, lymphoid cells deficient in NFATp and NFAT4 are isolated from the NFATp/NFAT4 deficient mouse, and are contacted with the test compound ex vivo to identify a test compound that modulates Th2 cell activity. In preferred embodiments, Th2 cell activity of the lymphoid cells deficient in NFATp and NFAT4 is inhibited by the test compound (thereby counteracting the increased Th2 cell activity caused by the NFATp/NFAT4 deficiency). Cells deficient in NFATp NFAT4 can be obtained from a mouse created to be deficient in NFATp and NFAT4.

In one embodiment of the screening assay, compounds tested for their ability to modulate Th2 cell activity are contacted with NFATp and NFAT4 deficient lymphoid cells by administering the test compound to an NFATp/NFAT4 deficient mouse in vivo and evaluating the effect of the test compound on Th2 cell activity in the mouse. The test compound can be administered to an NFATp/NFAT4 deficient mouse as a pharmaceutical composition. Such compositions typically comprise the test compound and a pharmaceutically acceptable carrier. As used herein the term “pharmaceutically acceptable carrier” is intended to include any and all solvents, dispersion media, coatings, antibacterial and antifungal compounds, isotonic and absorption delaying compounds, and the like, compatible with pharmaceutical administration. The use of such media and compounds for pharmaceutically active substances is well known in the art. Except insofar as any conventional media or compound is incompatible with the active compound, use thereof in the compositions is contemplated. Supplementary active compounds can also be incorporated into the compositions.

A pharmaceutical composition of the invention is formulated to be compatible with its intended route of administration. For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial compounds such as benzyl alcohol or methyl parabens; antioxidants such as ascorbic acid or sodium bisulfite; chelating compounds such as ethylenediarninetetraacetic acid; buffers such as acetates, citrates or phosphates and compounds for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal compounds, for example, parabens, chlorobutanol, phenol, ascorbic acid, thimerosal, and the like. In many cases, it will be preferable to include isotonic compounds, for example, sugars, polyalcohols such as manitol, sorbitol, sodium chloride in the composition. Prolonged absorption of the injectable compositions can be brought about by including in the composition an compound which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding compounds, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating compound such as alginic acid, Primogel, or corn starch; a lubricant such as magnesium stearate or Sterotes; a glidant such as colloidal silicon dioxide; a sweetening compound such as sucrose or saccharin; or a flavoring compound such as peppermint, methyl salicylate, or orange flavoring.

In one embodiment, the test compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These may be prepared according to methods known to those skilled in the art, for example, as described in U.S. Pat. No. 4,522,811.

In another embodiment, compounds that modulate Th2 cell activity are identified by contacting lymphoid cells deficient in NFATp and NFAT4 ex vivo with one or more test compounds, and determining the effect of the test compound on Th2 cell activity. In one embodiment NFATp and NFAT4 deficient lymphoid cells contacted with a test compound ex vivo may be readministered to a subject (e.g., an NFATp and/or NFAT4 deficient subject).

For practicing the screening method ex vivo, lymphoid cells deficient in NFATp and NFAT4 can be isolated from an NFATp/NFAT4 deficient mouse by standard methods and incubated (i.e., cultured) in vitro with a test compound. Methods for isolating and culturing lymphoid cells from mice are well known in the art (e.g., methods for isolating splenic, lymph node and/or peripheral blood lymphoid cells).

Following contact of the NFATp/NFAT4 deficient lymphoid cells with a test compound (either ex vivo or in vivo), the effect of the test compound on Th2 cell activity can be determined by any one of a variety of suitable methods, including monitoring of Th2-associated cytokine production or IgG1 and/or IgE production. Examples of such methods are described in detail in the Examples. A test compound is identified as a modulator of Th2 cell activity based on its ability to modulate Th2 cell activity of NFATp/NFAT4 deficient lymphoid cells, as compared to an appropriate control (such as untreated cells or cells treated with a control compound, or carrier, that does not modulate Th2 cell activity).

B. Assays Using NFATp- and NFAT4-Containing Indicator Compositions

In another embodiment, the invention provides methods for identifying compounds that modulate Th2 cell activity using indicator compositions that include NFATp and NFAT4. As described in the Examples, NFATp and NFAT4 have been demonstrated to be repressors of Th2 cell activity. Accordingly, compounds that specifically modulate the activity of NFATp and NFAT4 can be identified, as described herein, and the effect of a selected test compound on Th2 cell activity can be evaluated.

Thus, another aspect of the invention pertains to screening assays for identifying compounds that modulate Th2 cell activity comprising, providing at least one indicator composition comprising NFATp protein and NFAT4 protein;

contacting the at least one indicator composition with each member of a library of test compounds;

selecting from the library of test compounds a compound of interest that modulates the activity of NFATp protein and NFAT4 protein; and

determining the effect of the compound of interest on Th2 cell activity to thereby identify a compound that modulates Th2 cell activity.

The indicator composition can be a cell that expresses NFATp and/or NFAT4 proteins, for example, a cell that naturally expressed NFATp and/or NFAT4 (e.g., a T cell) or, more preferably, a cell that has been engineered to express the NFATp and/or NFAT4 proteins by introducing into the cell an expression vector(s) encoding the NFATp and/or NFAT4 proteins. Alternatively, the indicator composition can be a cell-free composition that includes NFATp and/or NFAT4 (e.g., a cell extract from an NFATp- and/or NFAT4-expressing cell or a composition that includes purified NFATp and/or NFAT4 proteins, either natural NFATp and/or NFAT4 or recombinant NFATp and/or NFAT4). In one embodiment, the indicator composition includes an NFATp and/or an NFAT4 protein and at least one target molecule with which NFATp and/or NFAT4 interacts, and the ability of the test compound to modulate the interaction of the NFATp and/or NFAT4 protein with the target molecule(s) is monitored to thereby identify the test compound as a modulator of NFATp and/or NFAT4 activity.

In one embodiment, a single indicator composition that comprises both NFATp and NFAT4 is used, whereas in a more preferred embodiment, one indicator composition comprises NFATp and another indicator composition comprises NFAT4, to thereby allow one to separately assess the effect of the test compound on either NFATp or NFAT4. For example, a library of test compounds can be screened against an indicator composition expressing NFATp to identify and select test compounds that modulate NFATp activity and then those selected test compounds that modulate NFATp activity can be rescreened against another indicator composition that expresses NFAT4 to identify and select test compounds that modulate both NFATp and NFAT4.

In preferred embodiments, the indicator composition(s) comprises an indicator cell(s), wherein the indicator cell(s) comprises an NFATp protein, an NFAT4 protein and at least one reporter gene responsive to the NFATp protein and/or the NFAT4 protein. Preferably, the indicator cell(s) contains:

at least one recombinant expression vector encoding the NFATp protein and the NFAT4 protein; and

at least one vector comprising an NFATp-responsive regulatory element operatively linked a reporter gene and an NFAT4-responsive regulatory element operatively linked to a reporter gene; and

the screening method comprises:

a) contacting the indicator cell(s) with a test compound;

b) determining the level of expression of the reporter gene(s) in the indicator cell(s) in the presence of the test compound; and

c) comparing the level of expression of the reporter gene(s) in the indicator cell(s) in the presence of the test compound with the level of expression of the reporter gene(s) in the indicator cell(s) in the absence of the test compound to thereby select a compound of interest that modulates the activity of NFATp and NFAT4 protein.

Once a test compound is identified as modulating the activity of NFATp and NFAT4, the effect of the test compound on Th2 cell activity is then tested.

NFATp- and NFAT4-responsive elements that can be used in the reporter gene construct are known in the art and include, for example, upstream regulatory regions from cytokine genes such as the IL-2, IL-4, GM-CSF, and TNF-α genes. Examples of NFATp-responsive reporter gene constructs are described, for example, in PCT Publication WO 97/39721 by Glimcher et al.

A cell that has been engineered to express the NFATp protein and/or the NFAT4 protein can be produced by introducing into the cell an expression vector encoding the NFATp and/or NFAT4 protein. Recombinant expression vectors that can be used for expression of NFATp and NFAT4 proteins in the indicator cell(s) are known in the art. Typically the NFATp/NFAT4 cDNA is first introduced into a recombinant expression vector using standard molecular biology techniques. An NFATp/NFAT4 cDNA can be obtained, for example, by amplification using the polymerase chain reaction (PCR) or by screening an appropriate cDNA library. The nucleotide sequences of NFATp/NFAT4 cDNAs (e.g., mouse and human) are known in the art and can be used for the design of PCR primers that allow for amplification of a cDNA by standard PCR methods or for the design of a hybridization probe that can be used to screen a cDNA library using standard hybridization methods. The nucleotide and predicted amino acid sequences of a mammalian NFATp cDNA are disclosed in McCaffrey, P. G. et al. (1993) Science 262:750-754 (see also U.S. Pat. No. 5,656,452 by Rao and U.S. Pat. No. 5,612,455 by Hoey) and the nucleotide and predicted amino acid sequences of mammalian NFAT4 cDNA are disclosed in Masuda, E. S. et al. (1995) Mol. Cell. Biol. 15:2697-2706.

Following isolation or amplification of a NFATp/NFAT4 cDNA, the DNA fragment is introduced into an expression vector. As used herein, the term “vector” refers to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” or simply “expression vectors”. In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors such as viral vectors (e.g., replication defective retroviruses, adenoviruses and adeno-associated viruses), which serve equivalent functions.

The recombinant expression vectors of the invention comprise a nucleic acid in a form suitable for expression of the nucleic acid in a host cell, which means that the recombinant expression vectors include one or more regulatory sequences, selected on the basis of the host cells to be used for expression and the level of expression desired, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to includes promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cell, those which direct expression of the nucleotide sequence only in certain host cells (e.g., tissue-specific regulatory sequences) or those which direct expression of the nucleotide sequence only under certain conditions (e.g., inducible regulatory sequences).

It will be appreciated by those skilled in the art that the design of the expression vector may depend on such factors as the choice of the host cell to be transformed, the level of expression of protein desired, etc. When used in mammalian cells, the expression vector's control functions are often provided by viral regulatory elements. For example, commonly used promoters are derived from polyoma virus, adenovirus, cytomegalovirus and Simian Virus 40. Non-limiting examples of mammalian expression vectors include pCDM8 (Seed, B., (1987)Nature 329:840) and pMT2PC (Kaufman et al. (1987), EMBO J. 6:187-195). A variety of mammalian expression vectors carrying different regulatory sequences are commercially available. For constitutive expression of the nucleic acid in a mammalian host cell, a preferred regulatory element is the cytomegalovirus promoter/enhancer. Moreover, inducible regulatory systems for use in mammalian cells are known in the art, for example systems in which gene expression is regulated by heavy metal ions (see e.g., Mayo et al. (1982) Cell 29:99-108; Brinster et al. (1982) Nature 296:39-42; Searle et al. (1985) Mol. Cell. Biol. 5:1480-1489), heat shock (see e.g., Nouer et al. (1991) in Heat Shock Response, e.d. Nouer, L., CRC, Boca Raton, Fla., pp167-220), hormones (see e.g., Lee et al. (1981) Nature 294:228-232; Hynes et al. (1981) Proc. Natl. Acad Sci. USA 78:2038-2042; Klock et al. (1987) Nature 329:734-736; Israel & Kaufman (1989) Nucl. Acids Res. 17:2589-2604; and PCT Publication No. WO 93/23431), FK506-related molecules (see e.g., PCT Publication No. WO 94/18317) or tetracyclines (Gossen, M. and Bujard, H. (1992) Proc. Natl. Acad. Sci. USA 89:5547-5551; Gossen, M. et al. (1995) Science 268:1766-1769; PCT Publication No. WO 94/29442; and PCT Publication No. WO 96/01313). Still further, many tissue-specific regulatory sequences are known in the art, including the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916) and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the α-fetoprotein promoter (Campes and Tilghman (1989) Genes Dev. 3:537-546).

Vector DNA can be introduced into mammalian cells via conventional transfection techniques. As used herein, the various forms of the term “transfection” are intended to refer to a variety of art-recognized techniques for introducing foreign nucleic acid (e.g., DNA) into mammalian host cells, including calcium phosphate co-precipitation, DEAE-dextran-mediated transfection, lipofection, or electroporation. Suitable methods for transfecting host cells can be found in Sambrook et al. (Molecular Cloning: A Laboratory Manual, 2nd Edition, Cold Spring Harbor Laboratory press (1989)), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin and methotrexate. Nucleic acid encoding a selectable marker may be introduced into a host cell on a separate vector from that encoding a maf family protein or, more preferably, on the same vector. Cells stably transfected with the introduced nucleic acid can be identified by drug selection (e.g, cells that have incorporated the selectable marker gene will survive, while the other cells die).

In another embodiments, the indicator composition is a cell free composition. NFATp and/or NFAT4 expressed by recombinant methods in a host cells or culture medium can be isolated from the host cells, or cell culture medium using standard methods for protein purifying, for example, by ion-exchange chromatography, gel filtration chromatography, ultrafiltration, electrophoresis, and immunoaffinity purification with antibodies specific for NFATp/NFAT4 to produce NFATp/NFAT4 protein that can be used in a cell free composition. Alternatively, an extract of NFATp and/or NFAT4-expressing cells can be prepared for use as cell-free composition.

In one embodiment, compounds that specifically modulate NFATp and NFAT4 activity are identified based on their ability to modulate the interaction of NFATp and NFAT4 with a target molecule(s) to which NFATp and/or NFAT4 binds. The target molecule can be a protein, such as c-fos, c-jun, AP-1 or NIP45. Alternatively, the target can be a DNA sequence (i.e., an NFATp- and/or NFAT4-responsive element). Suitable assays are known in the art that allow for the detection of protein-protein interactions (e.g., immunoprecipitations, two-hybrid assays and the like) or that allow for the detection of interactions between a DNA binding protein with a target DNA sequence (e.g., electrophoretic mobility shift assays, DNAse I footprinting assays and the like). By performing such assays in the presence and absence of test compounds, these assays can be used to identify compounds that modulate (e.g., inhibit or enhance) the interaction of NFATp and/or NFAT4 with a target molecule(s).

In one embodiment, the amount of binding of NFATp and/or NFAT4 to the target molecule(s) in the presence of the test compound is greater than the amount of binding of the NFATp and/or NFAT4 to the target molecule(s) in the absence of the test compound, in which case the test compound is identified as a compound that enhances binding of NFATp and/or NFAT4. In another embodiment, the amount of binding of the NFATp and/or NFAT4 to the target molecule(s) in the presence of the test compound is less than the amount of binding of the NFATp and/or NFAT4 to the target molecule(s) in the absence of the test compound, in which case the test compound is identified as a compound that inhibits binding of NFATp and/or NFAT4.

In the methods of the invention for identifying test compounds that modulate an interaction between NFATp and/or NFAT4 proteins and a target molecule(s), the full NFATp or NFAT4 protein may be used in the method, or, alternatively, only portions of the NFATp or NFAT4 protein may be used. For example, an isolated NFAT Rel Homology Domain (RHD) (or a larger subregion of NFATp/NFAT4 that includes the RHD) can be used. The degree of interaction between NFATp/NFAT4 proteins and the target molecule(s) can be determined, for example, by labeling one of the proteins with a detectable substance (e.g., a radiolabel), isolating the non-labeled protein and quantitating the amount of detectable substance that has become associated with the non-labeled protein. The assay can be used to identify test compounds that either stimulate or inhibit the interaction between the NFATp/NFAT4 proteins and a target molecule(s). A test compound that stimulates the interaction between the NFATp/NFAT4 proteins and a target molecule(s) is identified based upon its ability to increase the degree of interaction between the NFATp/NFAT4 proteins and a target molecule(s) as compared to the degree of interaction in the absence of the test compound, whereas a test compound that inhibits the interaction between the NFATp/NFAT4 proteins and a target molecule(s) is identified based upon its ability to decrease the degree of interaction between the NFATp/NFAT4 proteins and a target molecule(s) as compared to the degree of interaction in the absence of the compound. Assay systems for identifying compounds that modulate SH2 domain-ligand interactions as described in U.S. Pat. No. 5,352,660 by Pawson, can be adapted to identifying test compounds that modulate NFATp/NFAT4 target molecule(s) interaction.

Recombinant expression vectors that can be used for expression of NFATp and/or NFAT4 in the indicator cell are known in the art (see discussions above). In one embodiment, within the expression vector the NFATp- and/or NFAT4-coding sequences are operatively linked to regulatory sequences that allow for constitutive expression of NFATp and/or NFAT4 in the indicator cell(s) (e g., viral regulatory sequences, such as a cytomegalovirus promoter/enhancer, can be used). Use of a recombinant expression vector that allows for constitutive expression of NFATp/NFAT4 in the indicator cell is preferred for identification of compounds that enhance or inhibit the activity of NFATp/NFAT4. In an alternative embodiment, within the expression vector the NFATp/NFAT4 coding sequences are operatively linked to regulatory sequences of the endogenous NFATp/NFAT4 gene (i.e., the promoter regulatory region derived from the endogenous gene). Use of a recombinant expression vector in which NFATp/NFAT4 expression is controlled by the endogenous regulatory sequences is preferred for identification of compounds that enhance or inhibit the transcriptional expression of NFATp/NFAT4.

A variety of reporter genes are known in the art and are suitable for use in the screening assays of the invention. Examples of suitable reporter genes include those which encode chloramphenicol acetyltransferase, beta-galactosidase, alkaline phosphatase or luciferase. Standard methods for measuring the activity of these gene products are known in the art.

A variety of cell types are suitable for use as an indicator cell in the screening assay. Preferably a cell line is used which expresses low levels of NFATp/NFAT4, such as human Jurkat T cell leukemia, murine T cell hybridoma BYDP, or COS cells.

In one embodiment, the level of expression of the reporter gene in the indicator cell in the presence of the test compound is higher than the level of expression of the reporter gene in the indicator cell in the absence of the test compound and the test compound is identified as a compound that stimulates the expression or activity of NFATp/NFAT4. In another embodiment, the level of expression of the reporter gene in the indicator cell in the presence of the test compound is lower than the level of expression of the reporter gene in the indicator cell in the absence of the test compound and the test compound is identified as a compound that inhibits the expression or activity of NFATp/NFAT4.

Alternative to the use of a reporter gene construct, compounds that modulate the expression or activity of NFATp/NFAT4 can be identified by using other “read-outs.” For example, an indicator cell(s) can be transfected with a NFATp/NFAT4 expression vector(s), incubated in the presence and in the absence of a test compound, and IL-2 cytokine production can be assessed by detecting cytokine mRNA (e.g., IL-2 mRNA) in the indicator cell(s) or cytokine secretion (i.e., IL-2 secretion) into the culture supernatant. Standard methods for detecting cytokine mRNA, such as reverse transcription-polymerase chain reaction (RT-PCR) are known in the art. Standard methods for detecting cytokine protein in culture supernatants, such as enzyme linked immunosorbent assays (ELISA) are also known in the art.

Once a test compound is identified that modulates NFATp and NFAT4 activity, by one of the variety of methods described hereinbefore, the selected test compound (or “compound of interest”) can then be further evaluated for its effect on Th2 cell activity, for example by contacting the compound of interest with lymphoid cells either in vivo (e.g., by administering the compound of interest to a subject) or ex vivo (e.g., by isolating lymphoid cells and contacting the isolated lymphoid cells with the compound of interest or, alternatively, by contacting the compound of interest with a lymphoid cell line) and determining the effect of the compound of interest on Th2 cell activity, as compared to an appropriate control (such as untreated cells or cells treated with a control compound, or carrier, that does not modulate Th2 cell activity). The effect of the test compound on Th2 cell activity can be determined as described above in subsection A (e.g., by monitoring an indicator of Th2 cell activity, such as production of Th2-associated cytokine(s) or levels of IgG1 and/or IgE).

A variety of test compounds can be evaluated using the screening assays described in subsections A and B above. In certain embodiments, the compounds to be tested can be derived from libraries (i.e., are members of a library of compounds). While the use of libraries of peptides is well established in the art, new techniques have been developed which have allowed the production of mixtures of other compounds, such as benzodiazepines (Bunin et al. (1992). J. Am. Chem. Soc. 114:10987; DeWitt et al. (1993). Proc. Natl. Acad. Sci. USA 90:6909) peptoids (Zuckermann. (1994). J. Med. Chem. 37:2678) oligocarbamates (Cho et al. (1993). Science. 261:1303-), and hydantoins (DeWitt et al. supra). An approach for the synthesis of molecular libraries of small organic molecules with a diversity of 104-105 as been described (Carell et al. (1994). Angew. Chem Int. Ed. Engl. 33:2059-; Carell et al. (1994) Angew. Chem. Int. Ed. Engl. 33:2061-).

The compounds of the present invention can be obtained using any of the numerous approaches in combinatorial library methods known in the art, including: biological libraries; spatially addressable parallel solid phase or solution phase libraries, synthetic library methods requiring deconvolution, the ‘one-bead one-compound’ library method, and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145). Other exemplary methods for the synthesis of molecular libraries can be found in the art, for example in: Erb et al. (1994). Proc. Natl. Acad. Sci. USA 91:11422-; Horwell et al (1996) Immunopharmacology 33:68-; and in Gallop et al. (1994); J. Med. Chem. 37:1233-.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc Natl Acad Sci USA 89:1865-1869) or on phage (Scott and Smith (1990) Science 249:386-390); (Devlin (1990) Science 249:404-406); (Cwirla et al. (1990) Proc. Natl. Acad. Sci. 87:6378-6382); (Felici (1991) J Mol. Biol. 222:301-310); In still another embodiment, the combinatorial polypeptides are produced from a cDNA library.

Exemplary compounds which can be screened for activity include, but are not limited to, peptides, nucleic acids, carbohydrates, small organic molecules, and natural product extract libraries.

III. Methods for Modulating Th2 Cell Activity

In another aspect, the invention features a method for modulating Th2 cell activity by contacting lymphoid cells with a modulator of NFATp and NFAT4 activity such that Th2 cell activity is modulated. The invention also allows for modulation of aberrant Th2 cell activity in a subject in vivo, by administering to the subject a therapeutically effective amount of a modulator of NFATp and NFAT4 activity such that aberrant Th2 cell activity in a subject is modulated. The term “subject” is intended to include living organisms in which an immune response can be elicited. Preferred subjects are mammals. Examples of subjects include humans, monkeys, dogs, cats, mice, rats cows, horses, goats, and sheep. Modulation of NFATp and NFAT4 activity, therefore, provides a means to regulate aberrant Th2 cell activity in various disease states. In one embodiment, for stimulation of Th2 cell activity, the modulator inhibits NFATp and NFAT4 activity (which normally serve to repress Th2 cell activity). In another embodiment, to inhibit Th2 cell activity, the modulator stimulates NFATp and NFAT4 activity.

Identification of compounds that modulate Th2 cell activity by modulating NFATp and NFAT4 activity allows for selective manipulation of Th2 cell activity in a variety of clinical situations using the modulatory methods of the invention. The stimulatory methods of the invention (i.e., methods that use a stimulatory agent) result in decreased Th2 cell activity, which is desirable in diseases or conditions in which Th2 activity is detrimental. In contrast, the inhibitory methods of the invention (i.e., methods that use an inhibitory agent) result in increased Th2 cell activity, which is desirable in diseases or conditions in which Th2 activity is beneficial. Thus, to treat a disorder wherein Th2 cell activity is beneficial, a inhibitory method of the invention is selected such that NFATp and NFAT4 activity is inhibited. Alternatively, to treat a disorder wherein Th2 cell activity is detrimenal, a stimulatory method of the invention is selected such that NFATp and NFAT4 activity is upregulated to thereby repress Th2 cell activity. Application of the modulatory methods of the invention to the treatment of a disorder may result in cure of the disorder, a decrease in the type or number of symptoms associated with the disorder, either in the long term or short term (i.e., amelioration of the condition) or simply a transient beneficial effect to the subject.

Numerous disorders involving Th2 cell activity have been identified and could benefit from regulation of NFATp and NFAT4 in the individual suffering from the disorder. Application of the immunomodulatory methods of the invention to such disorders is described in further detail below.

A. Inhibitory Compounds

Since inhibition of NFATp and NFAT4 activity is associated with increased Th2 cell activity, to enhance Th2 cell activity cells are contacted with an agent that inhibits NFATp and NFAT4 activity. Cells (e.g., lymphoid cells) may be contacted with the agent in vitro and then the cells can be administered to a subject or, alternatively, the agent may be administered to the subject. The methods of the invention using NFATp and NFAT4 inhibitory compounds can be used in the treatment of disorders in which upregulation of Th2 cell activity is desirable, such as in various autoimmune diseases. For example, in experimental allergic encephalomyelitis (EAE), stimulation of a Th2-type response by administration of IL-4 at the time of the induction of the disease diminishes the intensity of the autoimmune disease (Paul, W. E., et al. (1994) Cell 76:241-251). Furthermore, recovery of the animals from the disease has been shown to be associated with an increase in a Th2-type response as evidenced by an increase of Th2-specific cytokines (Koury, S. J., et al. (1992) J. Exp. Med. 176:1355-1364). Moreover, T cells that can suppress EAE secrete Th2-specific cytokines (Chen, C., et al. (1994) Immunity 1:147-154). Since stimulation of a Th2-type response in EAE has a protective effect against the disease, stimulation of Th2 cell activity in subjects with multiple sclerosis (for which EAE is a model) may be beneficial therapeutically.

Similarly, stimulation of a Th2-type response in type I diabetes in mice provides a protective effect against the disease. Indeed, treatment of NOD mice with IL-4 (which promotes a Th2 response) prevents or delays onset of type I diabetes that normally develops in these mice (Rapoport, M. J., et al. (1993) J. Exp. Med. 178:87-99). Thus, stimulation of Th2 cell activity in a subject suffering from or susceptible to diabetes may ameliorate the effects of the disease or inhibit the onset of the disease.

Yet another autoimmune disease in which stimulation of a Th2-type response may be beneficial is rheumatoid arthritis (RA). Studies have shown that patients with rheumatoid arthritis have predominantly Th1 cells in synovial tissue (Simon, A. K., et al., (1994) Proc. Natl. Acad. Sci. USA 91:8562-8566). By stimulating Th2 cell activity in a subject with RA, the detrimental Th1 response can be concomitantly downmodulated to thereby ameliorate the effects of the disease.

Inhibitory compounds of the invention can be, for example, intracellular binding molecules that act to specifically inhibit the expression or activity of NFATp and NFAT4. As used herein, the term “intracellular binding molecule” is intended to include molecules that act intracellularly to inhibit the expression or activity of a protein by binding to the protein or to a nucleic acid (e.g., an mRNA molecule) that encodes the protein. Examples of intracellular binding molecules, described in further detail below, include antisense nucleic acids, intracellular antibodies, peptidic compounds that inhibit the interaction of NFATp and/or NFAT4 with a target molecule (e.g., calcineurin) and chemical agents that specifically inhibit NFATp and/or NFAT4 activity.

i. Antisense Nucleic Acid Molecules

In one embodiment, an inhibitory compound of the invention is an antisense nucleic acid molecule that is complementary to a gene encoding NFATp or NFAT4, or to a portion of said gene, or a recombinant expression vector encoding said antisense nucleic acid molecule. The use of antisense nucleic acids to downregulate the expression of a particular protein in a cell is well known in the art (see e.g., Weintraub, H. et al., Antisense RNA as a molecular tool for genetic analysis, Reviews—Trends in Genetics, Vol. 1(1) 1986; Askari, F. K. and McDonnell, W. M. (1996) N. Eng. J. Med. 334:316-318; Bennett, M. R. and Schwartz, S. M. (1995) Circulation 92:1981-1993; Mercola, D. and Cohen, J. S. (1995) Cancer Gene Ther. 2:47-59; Rossi, J. J. (1995) Br. Med. Bull. 51:217-225; Wagner, R. W. (1994) Nature 372:333-335). An antisense nucleic acid molecule comprises a nucleotide sequence that is complementary to the coding strand of another nucleic acid molecule (e.g., an mRNA sequence) and accordingly is capable of hydrogen bonding to the coding strand of the other nucleic acid molecule. Antisense sequences complementary to a sequence of an mRNA can be complementary to a sequence found in the coding region of the mRNA, the 5′ or 3′ untranslated region of the mRNA or a region bridging the coding region and an untranslated region (e.g., at the junction of the 5′ untranslated region and the coding region). Furthermore, an antisense nucleic acid can be complementary in sequence to a regulatory region of the gene encoding the mRNA, for instance a transcription initiation sequence or regulatory element. Preferably, an antisense nucleic acid is designed so as to be complementary to a region preceding or spanning the initiation codon on the coding strand or in the 3′ untranslated region of an mRNA.

Given the known nucleotide sequences for the coding strands of the NFATp and NFAT4 genes (and thus the known sequences of the NFATp and NFAT4 mRNAs), antisense nucleic acids of the invention can be designed according to the rules of Watson and Crick base pairing. The antisense nucleic acid molecule can be complementary to the entire coding region of a NFATp or NFAT4 mRNA, but more preferably is an oligonucleotide which is antisense to only a portion of the coding or noncoding region of a NFATp or NFAT4 mRNA. For example, the antisense oligonucleotide can be complementary to the region surrounding the translation start site of a NFATp or NFAT4 mRNA. An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. To inhibit NFATp and NFAT4 expression in cells in culture, one or more antisense oligonucleotides can be added to cells in culture media.

Alternatively, an antisense nucleic acid can be produced biologically using an expression vector into which all or a portion of NFATp or NFAT4 cDNA has been subcloned in an antisense orientation (i.e., nucleic acid transcribed from the inserted nucleic acid will be of an antisense orientation to a target nucleic acid of interest). Regulatory sequences operatively linked to a nucleic acid cloned in the antisense orientation can be chosen which direct the expression of the antisense RNA molecule in a cell of interest, for instance promoters and/or enhancers or other regulatory sequences can be chosen which direct constitutive, tissue specific or inducible expression of antisense RNA. The antisense expression vector is prepared according to standard recombinant DNA methods for constructing recombinant expression vectors, except that the NFATp or NFAT4 cDNA (or portion thereof) is cloned into the vector in the antisense orientation. The antisense expression vector can be in the form of, for example, a recombinant plasmid, phagemid or attenuated virus. The antisense expression vector is introduced into cells using a standard transfection technique.

The antisense nucleic acid molecules of the invention are typically administered to a subject or generated in situ such that they hybridize with or bind to cellular mRNA and/or genomic DNA encoding a NFATp or NFAT4 protein to thereby inhibit expression of the protein, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of an antisense nucleic acid molecule of the invention includes direct injection at a tissue site. Alternatively, an antisense nucleic acid molecule can be modified to target selected cells and then administered systemically. For example, for systemic administration, an antisense molecule can be modified such that it specifically binds to a receptor or an antigen expressed on a selected cell surface, e.g., by linking the antisense nucleic acid molecule to a peptide or an antibody which binds to a cell surface receptor or antigen. The antisense nucleic acid molecule can also be delivered to cells using the vectors described herein. To achieve sufficient intracellular concentrations of the antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In still another embodiment, an antisense nucleic acid of the invention is a ribozyme. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haselhoff and Gerlach (1988) Nature 334:585-591)) can be used to catalytically cleave NFATp or NFAT4 mRNA transcripts to thereby inhibit translation of NFATp or NFAT4 mRNAs. A ribozyme having specificity for a NFATp- or NFAT4-encoding nucleic acid can be designed based upon the nucleotide sequence of the NFATp or NFAT4 cDNA. For example, a derivative of a Tetrahymena L-19 IVS RNA can be constructed in which the nucleotide sequence of the active site is complementary to the nucleotide sequence to be cleaved in a NFATp- or NFAT4-encoding mRNA. See, e.g., Cech et al. U.S. Pat. No. 4,987,071 and Cech et al. U.S. Pat. No. 5,116,742. Alternatively, NFATp and NFAT4 mRNA can be used to select a catalytic RNA having a specific ribonuclease activity from a pool of RNA molecules. See, e.g., Bartel, D. and Szostak, J. W. (1993) Science 261:1411-1418.

Alternatively, NFATp and NFAT4 gene expression can be inhibited by targeting nucleotide sequences complementary to a regulatory region of an NFATP gene or NFAT4 gene (e.g., an NFATP or NFAT4 promoter and/or enhancer) to form triple helical structures that prevent transcription of an NFATP gene in target cells. See generally, Helene, C. (1991) Anticancer Drug Des. 6(6):569-84; Helene, C. et al. (1992) Ann. N. Y Acad. Sci. 660:27-36; and Maher, L. J. (1992) Bioassays 14(12):807-15.

ii. Intracellular Antibodies

Another type of inhibitory compound that can be used to inhibit the expression and/or activity of NFATp or NFAT4 protein in a cell is an intracellular antibody specific for NFATp or NFAT4 discussed herein. The use of intracellular antibodies to inhibit protein function in a cell is known in the art (see e.g., Carlson, J. R. (1988) Mol. Cell. Biol. 8:2638-2646; Biocca, S. et al. (1990) EMBO J. 9:101-108; Werge, T. M. et al. (1990) FEBS Letters 274:193-198; Carlson, J. R. (1993) Proc. Natl. Acad. Sci. USA 90:7427-7428; Marasco, W. A. et al. (1993) Proc. Natl. Acad. Sci. USA 90:7889-7893; Biocca, S. et al. (1994) Bio/Technology 12:396-399; Chen, S-Y. et al. (1994) Human Gene Therapy 5:595-601; Duan, L et al. (1994) Proc. Natl. Acad. Sci. USA 91:5075-5079; Chen, S-Y. et al. (1994) Proc. Natl. Acad. Sci. USA 91:5932-5936; Beerli, R. R. et al. (1994) J. Biol. Chem 269:23931-23936; Beerli, R. R. et al. (1994) Biochem. Biophys. Res. Commun. 204:666-672; Mhashilkar, A. M. et al. (1995) EMBO J. 14:1542-1551; Richardson, J. H. et al. (1995) Proc. Natl. Acad. Sci. USA 92:3137-3141; PCT Publication No. WO 94/02610 by Marasco et al.; and PCT Publication No. WO 95/03832 by Duan et al.).

To inhibit protein activity using an intracellular antibody, a recombinant expression vector is prepared which encodes the antibody chains in a form such that, upon introduction of the vector into a cell, the antibody chains are expressed as a functional antibody in an intracellular compartment of the cell. For inhibition of transcription factor activity according to the inhibitory methods of the invention, preferably an intracellular antibody that specifically binds the transcription factor is expressed within the nucleus of the cell. Nuclear expression of an intracellular antibody can be accomplished by removing from the antibody light and heavy chain genes those nucleotide sequences that encode the N-terminal hydrophobic leader sequences and adding nucleotide sequences encoding a nuclear localization signal at either the N- or C-terminus of the light and heavy chain genes (see e.g., Biocca, S. et al. (1990) EMBO J. 9:101-108; Mhashilkar, A. M. et al. (1995) EMBO J. 14:1542-1551). A preferred nuclear localization signal to be used for nuclear targeting of the intracellular antibody chains is the nuclear localization signal of SV40 Large T antigen (see Biocca, S. et al. (1990) EMBO J. 9:101-108; Mhashilkar, A. M. et al. (1995) EMBO J. 14:1542-1551).

To prepare an intracellular antibody expression vector, antibody light and heavy chain cDNAs encoding antibody chains specific for the target protein of interest, e.g., NFATp or NFAT4 protein, is isolated, typically from a hybridoma that secretes a monoclonal antibody specific for NFATp or NFAT4 protein. Preparation of antisera against NFATp or NFAT4 protein has been described in the art (see e.g., Rao et al, U.S. Pat. No. 5,656,452). Anti-NFATp or anti-NFAT4 antibodies can be prepared by immunizing a suitable subject, (e.g., rabbit, goat, mouse or other mammal) with a NFATp or NFAT4 immunogen. An appropriate immunogenic preparation can contain, for examples, recombinantly expressed NFATp or NFAT4 protein or a chemically synthesized NFATp or NFAT4 peptide. The preparation can further include an adjuvant, such as Freund's complete or incomplete adjuvant, or similar immunostimulatory compound. Antibody-producing cells can be obtained from the subject and used to prepare monoclonal antibodies by standard techniques, such as the hybridoma technique originally described by Kohler and Milstein (1975, Nature 256:495-497) (see also, Brown et al. (1981) J. Immunol 127:539-46; Brown et al. (1980) J. Biol Chem 255:4980-83; Yeh et al. (1976) PNAS 76:2927-31; and Yeh et al (1982) Int. J. Cancer 29:269-75). The technology for producing monoclonal antibody hybridomas is well known (see generally R. H. Kenneth, in Monoclonal Antibodies: A New Dimension In Biological Analyses, Plenum Publishing Corp., New York, N.Y. (1980); E. A. Lemer (1981) Yale J. Biol. Med., 54:387-402; M. L. Gefter et al. (1977) Somatic Cell Genet., 3:231-36). Briefly, an immortal cell line (typically a myeloma) is fused to lymphocytes (typically splenocytes) from a mammal immunized with a NFATp or NFAT4 protein immunogen as described above, and the culture supernatants of the resulting hybridoma cells are screened to identify a hybridoma producing a monoclonal antibody that binds specifically to the NFATp or NFAT4 protein. Any of the many well known protocols used for fusing lymphocytes and immortalized cell lines can be applied for the purpose of generating an anti-NFATp or NFAT4 protein monoclonal antibody (see, e.g., G. Galfre et al. (1977) Nature 266:550-52; Gefter et al. Somatic Cell Genet., cited supra; Lerner, Yale J. Biol. Med., cited supra; Kenneth, Monoclonal Antibodies, cited supra). Moreover, the ordinary skilled artisan will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines may be used as a fusion partner according to standard techniques, e.g., the P3-NSI/1-Ag4-1, P3-x63-Ag8.653 or Sp2/O-Ag14 myeloma lines. These myeloma lines are available from the American Type Culture Collection (ATCC), Rockville, Md. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody that specifically binds the maf protein are identified by screening the hybridoma culture supernatants for such antibodies, e.g., using a standard ELISA assay.

Alternative to preparing monoclonal antibody-secreting hybridomas, a monoclonal antibody that binds to NFATp or NFAT4 can be identified and isolated by screening a recombinant combinatorial immunoglobulin library (e.g., an antibody phage display library) with the protein, or a peptide thereof, to thereby isolate immunoglobulin library members that bind specifically to the protein. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and compounds particularly amenable for use in generating and screening antibody display library can be found in, for example, Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Fuchs et al. (1991) Bio/Technology 9:1370-1372; Hay et al. (1992) Hum Antibod Hybridomas 3:81-85; Huse et al. (1989) Science 246:1275-1281; Griffiths et al. (1993) EMBO J. 12:725-734; Hawkins et al. (1992) J. Mol Biol 226:889-896; Clarkson et al. (1991) Nature 352:624-628; Gram et al. (1992) PNAS 89:3576-3580; Garrad et al (1991) Bio/Technology 9:1373-1377; Hoogenboom et al. (1991) Nuc Acid Res 19:4133-4137; Barbas et al (1991) PNAS 88:7978 7982; and McCafferty et al. Nature (1990) 348:552-554.

Once a monoclonal antibody of interest specific for NFATp and/or NFAT4 has been identified (e.g., either a hybridoma-derived monoclonal antibody or a recombinant antibody from a combinatorial library, including monoclonal antibodies to NFATp and/or NFAT4 that are already known in the art), DNAs encoding the light and heavy chains of the monoclonal antibody are isolated by standard molecular biology techniques. For hybridoma derived antibodies, light and heavy chain cDNAs can be obtained, for example, by PCR amplification or cDNA library screening. For recombinant antibodies, such as from a phage display library, cDNA encoding the light and heavy chains can be recovered from the display package (e.g., phage) isolated during the library screening process. Nucleotide sequences of antibody light and heavy chain genes from which PCR primers or cDNA library probes can be prepared are known in the art. For example, many such sequences are disclosed in Kabat, E. A., et al. (1991) Sequences of Proteins of Immunological Interest, Fifth Edition, U.S. Department of Health and Human Services, NIH Publication No. 91-3242 and in the “Vbase” human germline sequence database.

Once obtained, the antibody light and heavy chain sequences are cloned into a recombinant expression vector using standard methods. As discussed above, the sequences encoding the hydrophobic leaders of the light and heavy chains are removed and sequences encoding a nuclear localization signal (e.g., from SV40 Large T antigen) are linked in-frame to sequences encoding either the amino- or carboxy terminus of both the light and heavy chains. The expression vector can encode an intracellular antibody in one of several different forms. For example, in one embodiment, the vector encodes full-length antibody light and heavy chains such that a full-length antibody is expressed intracellularly. In another embodiment, the vector encodes a full-length light chain but only the VH/CH1 region of the heavy chain such that a Fab fragment is expressed intracellularly. In the most preferred embodiment, the vector encodes a single chain antibody (scFv) wherein the variable regions of the light and heavy chains are linked by a flexible peptide linker (e.g., (Gly₄Ser)₃) and expressed as a single chain molecule. To inhibit transcription factor activity in a cell, the expression vector encoding the NFATp- and/or NFAT4-specific intracellular antibody is introduced into the cell by standard transfection methods as described hereinbefore.

iii. NFATp-Derived Peptidic Compounds

In another embodiment, an inhibitory compound of the invention is a peptidic compound derived from the NFATp and/or NFAT4 amino acid sequence. In particular, the inhibitory compound(s) comprises a portion of NFATp and/or NFAT4 (or a mimetic thereof) that mediates interaction of NFATp/NFAT4 with a target molecule such that contact of NFATp/NFAT4 with this peptidic compound competitively inhibits the interaction of NFATp with the target molecule. In a preferred embodiment, the peptide compound is designed based on the region of NFATp/NFAT4 that mediates interaction of NFATp/NFAT4 with calcineurin. As described in Avramburu et al., (1998) Mol. Cell. 1:627-637 (expressly incorporated herein by reference), a conserved region in the amino terminus of NFAT proteins mediates interaction of the NFAT proteins with calcineurin and peptides spanning the region inhibit the ability of calcineurin to bind to and phosphorylate NFAT proteins, without affecting the phosphatase activity of calcineurin against other substrates. Moreover, when expressed intracellularly, peptide spanning this region inhibits NFAT dephosphorylation, nuclear translocation and NFAT-mediated gene expression in response to stimulation, thereby inhibiting NFAT-dependent functions. The region of NFATp mediating interaction with calcineurin contains the conserved amino acid motif: Ser-Pro-Arg-Ile-Glu-Ile-Thr (SEQ ID NO:1).

In a preferred embodiment, a NFAT inhibitory compound is a peptidic compound, which is prepared based on a calcineurin-interacting region of NFATp. A peptide can be derived from the calcineurin-interacting region of NFATp having an amino acid sequence that comprises the 9 amino acid motif of SEQ ID NO:1. Alternatively, longer regions of human NFATp can be used such as a peptide that comprises the 25 amino acids of SEQ ID NO: 2 (which spans the motif of SEQ ID NO: 1) or the 13 amino acids of SEQ ID NO: 3 (which also spans the motif of SEQ ID NO: 1).

The peptidic compounds of the invention can be made intracellularly in cells (e.g., lymphoid cells) by introducing into the cells an expression vector encoding the peptide(s). Such expression vectors can be made by standard techniques, using, for example, oligonucleotides that encode the amino acid sequences of SEQ ID NO: 1, SEQ ID NO: 2 or SEQ ID NO: 3. The peptide(s) can be expressed in intracellularly as a fusion with another protein or peptide (e.g., a GST fusion). Alternative to recombinant synthesis of the peptides in the cells, the peptides can be made by chemical synthesis using standard peptide synthesis techniques. Synthesized peptides can then be introduced into cells by a variety of means known in the art for introducing peptides into cells (e.g., liposome and the like). Recombinant methods of making NFAT inhibitory peptides, and methods using them to inhibit NFAT activity in cells, are described further in Avramburu et al., (1998) Mol. Cell. 1:627-637.

It also has been demonstrated that the region of NFATp that interacts with calcineurin is necessary for nuclear import of NFATp and for effective recognition and dephosphorylation such that mutation of this region inhibits NFATp activity (see Avramburu et al., (1998) Mol. Cell. 1:627-637). Thus, in another embodiment, NFATp activity can be inhibited by mutating the calcineurin-binding region in the amino terminus, comprising the motif of SEQ ID NO: 1. An example of a mutated sequence of this motif that with greatly reduced ability to interact with calcineurin is shown in SEQ ID NO: 4. The wildtype NFATp amino acid can be modified to the mutated sequence to create a mutated form of NFATp with reduced activity.

Other inhibitory agents that can be used to specifically inhibit the activity of NFATp and NFAT4 proteins are chemical compounds that directly inhibit NFATp and NFAT4 activity or inhibit the interaction between NFATp, NFAT4 and target molecules. Such compounds can be identified using screening assays that select for such compounds, as described in detail above.

B. Stimulatory Compounds

Since downregulation of NFATp and NFAT4 activity is associated with increased Th2 cell activity, a compound that specifically stimulates NFATp and NFAT4 activity can be used to inhibit Th2 cell activity. In the stimulatory methods of the invention, a subject is treated with a stimulatory compound that stimulates expression and/or activity of NFATp and NFAT4. The methods of the invention using NFATp and NFAT4 stimulatory compounds can be used in the treatment of disorders in which downregulation of Th2 cell activity is beneficial, such as allergies (to thereby downregulate IgE production) and infectious diseases and cancers, in which biasing of the immune response to a Th1 type response may be beneficial.

Examples of stimulatory compounds include active NFATp/NFAT4protein, expression vectors encoding NFATp/NFAT4 and chemical agents that specifically stimulate NFATp and NFAT4 activity.

A preferred stimulatory compound is at least one nucleic acid molecule encoding NFATp and NFAT4, wherein the nucleic acid molecule(s) is introduced into the subject in a form suitable for expression of the NFATp and NFAT4 proteins in the cells of the subject. For example, NFATp and NFAT4 cDNAs (full length or partial NFATp and NFAT4 cDNA sequence) is cloned into a recombinant expression vector and the vector is transfected into cells using standard molecular biology techniques. The NFATp and NFAT4 cDNAs can be obtained, for example, by amplification using the polymerase chain reaction (PCR) or by screening an appropriate cDNA library. The nucleotide sequences of NFATp and NFAT 4 cDNAs are known in the art and can be used for the design of PCR primers that allow for amplification of the cDNAs by standard PCR methods or for the design of a hybridization probe that can be used to screen a cDNA library using standard hybridization methods.

Following isolation or amplification of NFATp and NFAT4 cDNAs, the DNA fragments are introduced into one or more suitable expression vector, as described above. A single expression vector that carries both NFATp and NFAT4 coding sequences can be used or two separate vectors, one encoding NFATp and the other encoding NFAT4, can be used. Nucleic acid molecules encoding NFATp and NFAT4 in the form suitable for expression of the NFATp and NFAT4 in a host cell, can be prepared as described above using nucleotide sequences known in the art. The nucleotide sequences can be used for the design of PCR primers that allow for amplification of a cDNA by standard PCR methods or for the design of a hybridization probe that can be used to screen a cDNA library using standard hybridization methods.

Another form of a stimulatory compound for stimulating expression of NFATp and NFAT4 in a cell is a chemical compound that specifically stimulates the expression or activity of endogenous NFATp and NFAT4 in the cell. Such compounds can be identified using screening assays that select for compounds that stimulate the expression or activity of NFATp and NFAT4 as described herein.

The method of the invention for modulating aberrant cartilage growth in a subject can be practiced either in vitro or in vivo (the latter is discussed further in the following subsection). For practicing the method in vitro, cells can be obtained from a subject by standard methods and incubated (i.e., cultured) in vitro with a stimulatory or inhibitory compound of the invention to stimulate or inhibit, respectively, the activity of NFATp and NFAT4.

Cells treated in vitro with either a stimulatory or inhibitory compound can be administered to a subject to influence Th2 cell activity in the subject. For example, lymphoid cells can be isolated from a subject, treated in vitro using a modulatory agent of the invention and then readministered to the same subject, or another subject tissue compatible with the donor of the cells. Accordingly, in another embodiment, the modulatory method of the invention comprises culturing cells in vitro with a NFATp/NFAT4 modulator and further comprises administering the cells to a subject to thereby modulate Th2 cell activity in a subject. For administration of cells to a subject, it may be preferable to first remove residual compounds in the culture from the cells before administering them to the subject. This can be done for example by gradient centrifugation of the cells or by washing of the cells. For further discussion of ex vivo genetic modification of cells followed by readministration to a subject, see also U.S. Pat. No. 5,399,346 by W. F. Anderson et al.

In other embodiments, a stimulatory or inhibitory compound is administered to a subject in vivo. For stimulatory or inhibitory agents that comprise nucleic acids (e.g., recombinant expression vectors encoding NFATp/NFAT4, antisense RNA, intracellular antibodies or NFATp- or NFAT4-derived peptides), the compounds can be introduced into cells of a subject using methods known in the art for introducing nucleic acid (e.g., DNA) into cells in vivo. Examples of such methods include:

Direct Injection:

Naked DNA can be introduced into cells in vivo by directly injecting the DNA into the cells (see e.g., Acsadi et al. (1991) Nature 332:815-818; Wolff et al. (1990) Science 247:1465-1468). For example, a delivery apparatus (e.g., a “gene gun”) for injecting DNA into cells in vivo can be used. Such an apparatus is commercially available (e.g., from BioRad).

Receptor-Mediated DNA Uptake:

Naked DNA can also be introduced into cells in vivo by complexing the DNA to a cation, such as polylysine, which is coupled to a ligand for a cell-surface receptor (see for example Wu, G. and Wu, C. H. (1988) J. Biol. Chem. 263:14621; Wilson et al. (1992) J. Biol. Chem. 267:963-967; and U.S. Pat. No. 5,166,320). Binding of the DNA-ligand complex to the receptor facilitates uptake of the DNA by receptor-mediated endocytosis. A DNA-ligand complex linked to adenovirus capsids which naturally disrupt endosomes, thereby releasing material into the cytoplasm can be used to avoid degradation of the complex by intracellular lysosomes (see for example Curiel et al. (1991) Proc. Natl. Acad. Sci. USA 88:8850; Cristiano et al. (1993) Proc. Natl. Acad. Sci. USA 90:2122-2126).

Retroviruses:

Defective retroviruses are well characterized for use in gene transfer for gene therapy purposes (for a review see Miller, A. D. (1990) Blood 76:271). A recombinant retrovirus can be constructed having a nucleotide sequences of interest incorporated into the retroviral genome. Additionally, portions of the retroviral genome can be removed to render the retrovirus replication defective. The replication defective retrovirus is then packaged into virions which can be used to infect a target cell through the use of a helper virus by standard techniques. Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14 and other standard laboratory manuals. Examples of suitable retroviruses include pLJ, pZIP, pWE and pEM which are well known to those skilled in the art. Examples of suitable packaging virus lines include ΨCrip, ΨCre, Ψ2 and ΨAm. Retroviruses have been used to introduce a variety of genes into many different cell types, including epithelial cells, endothelial cells, lymphocytes, myoblasts, hepatocytes, bone marrow cells, in vitro and/or in vivo (see for example Eglitis, et al. (1985) Science 230:1395-1398; Danos and Mulligan (1988) Proc. Nail. Acad Sci. USA 85:6460-6464; Wilson et al. (1988) Proc. Nail Acad Sci. USA 85:3014-3018; Armentano et al. (1990) Proc. Natl. Acad Sci. USA 87:6141-6145; Huber et al. (1991) Proc. Natl. Acad. Sci. USA 88:8039-8043; Ferry et al. (1991) Proc. Natl. Acad Sci. USA 88:8377-8381; Chowdhury et al. (1991) Science 254:1802-1805; van Beusechem et al. (1992) Proc. Natl. Acad Sci. USA 89:7640-7644; Kay et al. (1992) Human Gene Therapy 3:641-647; Dai et al (1992) Proc. Natl. Acad. Sci. USA 89:10892-10895; Hwu et al. (1993)J. Immunol. 150:4104-4115; U.S. Pat. No. 4,868,116; U.S. Pat. No. 4,980,286; PCT Application WO 89/07136; PCT Application WO 89/02468; PCT Application WO 89/05345; and PCT Application WO 92/07573). Retroviral vectors require target cell division in order for the retroviral genome (and foreign nucleic acid inserted into it) to be integrated into the host genome to stably introduce nucleic acid into the cell. Thus, it may be necessary to stimulate replication of the target cell.

Adenoviruses:

The genome of an adenovirus can be manipulated such that it encodes and expresses a gene product of interest but is inactivated in terms of its ability to replicate in a normal lytic viral life cycle. See for example Berkner et al. (1988) BioTechniques 6:616; Rosenfeld et al. (1991) Science 252:431-434; and Rosenfeld et al. (1992) Cell 68:143-155. Suitable adenoviral vectors derived from the adenovirus strain Ad type 5 dl324 or other strains of adenovirus (e.g., Ad2, Ad3, Ad7 etc.) are well known to those skilled in the art. Recombinant adenoviruses are advantageous in that they do not require dividing cells to be effective gene delivery vehicles and can be used to infect a wide variety of cell types, including airway epithelium (Rosenfeld et al. (1992) cited supra), endothelial cells (Lemarchand et al. (1992) Proc. Natl. Acad. Sci. USA 89:6482-6486), hepatocytes (Herz and Gerard (1993) Proc. Natl. Acad. Sci. USA 90:2812-2816) and muscle cells (Quantin et al. (1992) Proc. Natl. Acad. Sci. USA 89:2581-2584). Additionally, introduced adenoviral DNA (and foreign DNA contained therein) is not integrated into the genome of a host cell but remains episomal, thereby avoiding potential problems that can occur as a result of insertional mutagenesis in situations where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al. cited supra; Haj-Ahmand and Graham (1986) J. Virol. 57:267). Most replication-defective adenoviral vectors currently in use are deleted for all or parts of the viral E1 and E3 genes but retain as much as 80% of the adenoviral genetic material.

Adeno-Associated Viruses:

Adeno-associated virus (AAV) is a naturally occurring defective virus that requires another virus, such as an adenovirus or a herpes virus, as a helper virus for efficient replication and a productive life cycle. (For a review see Muzyczka et al. Curr. Topics in Micro. and Immunol (1992) 158:97-129). It is also one of the few viruses that may integrate its DNA into non-dividing cells, and exhibits a high frequency of stable integration (see for example Flotte et al. (1992) Am. J. Respir. Cell. Mol. Biol. 7:349-356; Samulski et al. (1989) J. Virol. 63:3822-3828; and McLaughlin et al. (1989) J. Virol. 62:1963-1973). Vectors containing as little as 300 base pairs of AAV can be packaged and can integrate. Space for exogenous DNA is limited to about 4.5 kb. An AAV vector such as that described in Tratschin et al. (1985) Mol. Cell. Biol. 5:3251-3260 can be used to introduce DNA into cells. A variety of nucleic acids have been introduced into different cell types using AAV vectors (see for example Hermonat et al. (1984) Proc. Natl. Acad Sci. USA 81:6466-6470; Tratschin et al. (1985) Mol. Cell. Biol. 4:2072-2081; Wondisford et al (1988) Mol. Endocrinol. 2:32-39; Tratschin et al. (1984) J. Virol. 51:611-619; and Flotte et al. (1993) J. Biol. Chem. 268:3781-3790).

The efficacy of a particular expression vector system and method of introducing nucleic acid into a cell can be assessed by standard approaches routinely used in the art. For example, DNA introduced into a cell can be detected by a filter hybridization technique (e.g, Southern blotting) and RNA produced by transcription of introduced DNA can be detected, for example, by Northern blotting, RNase protection or reverse transcriptase-polymerase chain reaction (RT-PCR). The gene product can be detected by an appropriate assay, for example by immunological detection of a produced protein, such as with a specific antibody, or by a functional assay to detect a functional activity of the gene product, such as an enzymatic assay.

If the stimulatory or inhibitory compounds are chemical compounds that modulate NFATp and NFAT4 activity, the stimulatory or inhibitory compounds can be administered to a subject as a pharmaceutical composition. Such compositions typically comprise the stimulatory or inhibitory compounds and a pharmaceutically acceptable carrier. Pharmaceutically acceptable carriers and methods of administration to a subject are described above.

IV. Diagnostic Assays

In another aspect, the invention features a method of diagnosing a subject for a disorder associated with Th2 cell activity comprising:

(a) detecting expression of NFATp and NFAT4 in cells of a subject suspected of having a disorder associated with Th2 cell activity;

(b) comparing expression of NFATp and NFAT4 in cells of said subject to a control that is not associated with aberrant Th2 cell activity; and

(c) diagnosing the subject for a disorder based on a change in expression of NFATp or NFAT4 in cells of the subject as compared to the control.

The “change in expression of NFATp or NFAT4” in cells of the subject can be, for example, a change in the level of expression of NFATp or NFAT4 in cells of the subject, which can be detected by assaying levels of NFATp or NFAT4 mRNA, for example, by isolating cells from the subject and determining the level of NFATp or NFAT4 mRNA expression in the cells by standard methods known in the art, including Northern blot analysis, reverse-transcriptase PCR analysis and in situ hybridizations. Alternatively, the level of expression of NFATp or NFAT4 in cells of the subject can be detected by assaying levels of NFATp or NFAT4 protein, for example, by isolating cells from the subject and determining the level of NFATp or NFAT4 protein expression by standard methods known in the art, including Western blot analysis, immunoprecipitations, enzyme linked immunosorbent assays (ELISAs) and immunofluorescence.

In another embodiment, a change in expression of NFATp or NFAT4 in cells of the subject result from one or more mutations (i.e., alterations from wildtype) in the NFATp and/or NFAT4 gene and mRNA leading to one or more mutations (i.e., alterations from wildtype) in the amino acid sequence of the NFATp and/or NFAT4 protein. In one embodiment, the mutation(s) leads to a form of NFATp and/or NFAT4 with increased activity (e.g., partial or complete constitutive activity). In another embodiment, the mutation(s) leads to a form of NFATp and/or NFAT4 with decreased activity (e.g., partial or complete inactivity). The mutation(s) may change the level of expression of NFATp/NFAT4, for example, increasing or decreasing the level of expression of NFATp/NFAT4 in a subject with a disorder. Alternatively, the mutation(s) may change the regulation of NFATp/NFAT4, for example, by the interaction of the mutant NFATp/NFAT4 with upstream targets of NFATp/NFAT4, such as calcineurin. The mutation(s) may alter the ability of NFATp/NFAT4 to regulate downstream NFATp/NFAT4 targets, such as cytokines in a subject with a disorder. Mutations in the nucleotide sequence or amino acid sequences of NFATp/NFAT4 can be determined using standard techniques for analysis of DNA or protein sequences, for example for DNA or protein sequencing, RFLP analysis, and analysis of single nucleotide or amino acid polymorphisms.

In preferred embodiments, the diagnostic assay is conducted on a biological sample from the subject, such as a cell sample or a tissue section (for example, a freeze-dried or fresh frozen section of tissue removed from a subject). In another embodiment, the level of expression of NFATp and NFAT4 in cells of the subject can be detected in vivo, using an appropriate imaging method, such as using a radiolabeled anti-NFATp and anti-NFAT4 antibody.

In one embodiment, the level of expression of NFATp/NFAT4 in cells of the test subject may be elevated (i.e., increased) relative to the control not associated with the disorder or the subject may express a constitutively active (partially or completely) form of NFATp/NFAT4. This elevated expression level of NFATp/NFAT4 or expression of a constitutively active form of NFATp/NFAT4 can be used to diagnose a subject for a disorder associated with decreased Th2 cell activity. In another embodiment, the level of expression of NFATp/NFAT4 in cells of the subject may reduced (i.e., decreased) relative to the control not associated with the disorder or the subject may express an inactive (partially or completely) mutant form of NFATp/NFAT4. This reduced expression level of NFATp/NFAT4 or expression of an inactive mutant form of NFATp/NFAT4 can be used to diagnose a subject for a disorder associated with increased Th2 cell activity.

V. Kits of the Invention

Another aspect of the invention pertains to kits for carrying out the screening assays, modulatory methods or diagnostic assays of the invention. For example, a kit for carrying out a screening assay of the invention can include a NFATp and NFAT4 doubly deficient mouse, or NFATp and NFAT4 doubly deficient cells thereof, means for determining Th2 cell activity and instructions for using the kit to identify modulators of Th2 cell activity. In another embodiment, a kit for carrying out a screening assay of the invention can include an indicator composition comprising NFATp and NFAT4 proteins, means for determining Th2 cell activity and instructions for using the kit to identify modulators of Th2 cell activity.

In another embodiment, the invention provides a kit for carrying out a modulatory method of the invention. The kit can include, for example, a modulatory agent of the invention (e.g., NFATp/NFAT4 inhibitory or stimulatory agent) in a suitable carrier and packaged in a suitable container with instructions for use of the modulator to modulate Th2 cell activity.

Another aspect of the invention pertains to a kit for diagnosing a disorder associated with aberrant Th2 cell activity in a subject. The kit can include a reagent for determining expression of NFATp and NFAT4 (e.g., a nucleic acid probe(s) for detecting NFATp and NFAT4 mRNA or one or more antibodies for detection of NFATp and NFAT4 proteins), a control to which the results of the subject are compared, and instructions for using the kit for diagnostic purposes.

This invention is further illustrated by the following examples which should not be construed as limiting. The contents of all references, patents and published patent applications cited throughout this application are hereby incorporated by reference.

### EXAMPLE 1

### Preparation and Characterization of Mice Doubly Deficient in NFATp and NFAT4

Examination of NFAT family member expression in T helper clones by Northern blot analysis upon activation revealed a rapid induction of mRNA transcripts encoding NFATc concomitant with a downregulation of both NFATp and NFAT4 expression. Blots were hybridized with cDNA probes encoding the NFATc, NFAT4 and NFATp genes [Hodge, M. R. et al, Immunity 4:1 (1996); Hoey, T. et al., Immunity 2:461 (1995). This result raised the possibility that NFATc acts as an activator of, and NFAT4 and NFATp as repressors of, the immune response.

Mice lacking NFATc in the lymphoid system (as evaluated by RAG-2 blastocyst complementation) have mildly impaired proliferation and a selective decrease in IL-4 production [Ranger, A. M. et al., Immunity 8:125 (1998); Yoshida, H. et al., Immunity 8:115 (1998)] consistent with a function of NFATc as a positive regulator of the immune system. Conversely, mice lacking NFATp [described in [Hodge, M. R. et al., Immunity 4:1 (1996); Xanthoudakis, S. et al., Science 272:892 (1996)] display modest splenomegaly, T and B cell hyperproliferation and cytokine dysregulation during the course of an immune response with a moderate increase in Th2-type cytokines. Mice lacking NFAT4 have normal peripheral T cell proliferation and cytokine production although an increased percentage (390%) of T and B cells display a phenotype characteristic of memory/activated cells [Oukka, M. et al., Immunity 9:295-304 (1998)] The modest inhibitory effects of the single NFATp and NFAT4 gene deletions, however, suggested either that the repressive effect of each was independent but not profound, or that these proteins were functionally redundant.

To test these hypotheses, we intercrossed NFATp and NFAT4 null mice to generate mice doubly deficient in these two NFAT proteins (DKO). NFATp-deficient mice can be prepared, for example, as described in Hodge, M.R. et al., Immunity 4:1 (1996) and NFAT4-deficient mice can be prepared, for example, as described in Oukka, M. et al., Immunity 9:295-304 (1998). Doubly-deficient mice can be obtained by standard cross-breeding of the singly-deficient animals.

DKO mice demonstrated modest growth retardation and developed severe bilateral blepharitis by approximately 4 weeks after birth. Histological evaluation of the eye and the surrounding tissues revealed a complex cellular infiltrate composed of lymphocytes, macrophages, mast cells and plasma cells. In all DKO animals examined (n=5), the eyelids displayed edema and ulceration with underlying granulation tissue and a marked inflammatory infiltrate. Examination of the lungs revealed an acute and chronic interstitial pneumonitis characterized by an intense inflammatory infiltrate consisting of lymphocytes, plasma cells, neutrophils and mast cells or basophils. The inflammatory infiltrate suprisingly did not include eosinophils. There was no evidence of inflammatory disease in the heart, kidney or liver and no evidence of renal or pancreatic dysfunctional s judged by the absence of urinary glucose and protein.

DKO mice exhibited massive splenomegaly and lymphadenopathy by 7 weeks of age. Histological analysis of the spleen and lymph node revealed disruption of the normal architecture by numerous granulomas. The architecture of the lymph node and spleen of the DKO is disrupted by granulomatous lesions containing multinucleated giant cells. There was also a marked increase in mast cells in DKO spleen. Toluidine-blue stained spleen sections from wild-type and DKO mice showed numerous mast cells identified by intense staining of intracellular granules. The absence of multiorgan lymphoid infiltration and immune complex-mediated pathology distinguishes the NFAT DKO from other mouse strains that display massive lymphadenopathy such as CTLA-4 and IL-2 receptor alpha deficient and TRAF2 dominant negative mutant mice [Tivol, E. A. et al., Immunity 3:541 (1995); Waterhosue, P. et al., Science 270:985 (1995); Sadlack, B. et al., Eur. J. Immunol. 25:3053 (1995); Willerford, D. M. et al., Immunity 3:521 (1995)]. This is consistent with the normal protein or RNA levels of these genes in NFAT DKO lymphocytes.

In contrast to the increased size of the peripheral lymphoid organs, the thymus was somewhat (50-70%) smaller than wild-type at 7-14 weeks. The composition was slightly abnormal with increased numbers of SP thymocytes, possibly the result of infiltration of mature T and B cells from the periphery as seen in CTLA4 null mice or secondary to elevated levels of IL-4 [Tepper, R. I. et al., Cell 62:457 (1990)] (see below).

Flow cytometric analysis of peripheral lymphoid organs was performed on lymphocytes from wild type and DKO mice from thymus, spleen and lymph node. Single cell suspensions were stained with the following antibodies: anti-CD4-TC, anti-CD8-PE, anti-B220-PE and anti-CD3-FITC. Flow cytometric analysis of peripheral lymphoid organs revealed a modest increase in the percentage of B220+cells and a corresponding decrease in CD3+T cells in both the spleen and LN. The ratio of CD4+/CD8+T cells was also skewed with an increased percentage of CD8+T cells in LN and a substantial decrease in the spleen. An increased number of non-T, non-B cells of unclear identity were present.

In the absence of NFATp and NFAT4 there was a dramatic increase in the percentage of peripheral T cells with a memory/activated phenotype as indicated by low levels of Mel-14 and CD45RB and elevated levels of CD44 and CD69 on spleen cells and LN. The activated/memory cells did not represent a clonal expansion of T cells as evaluated by their Vβ and Vα usage. DKO B cells were also hyperactivated as demonstrated by upregulation of MHC Class II and increased numbers of IgM-negative B220+ cells.

### EXAMPLE 2

### NFATp/NFAT4 Doubly-Deficient Mice Exhibit Compromised Fas Ligand Expression

The massive lymphadenopathy in the DKO mice could potentially be explained by increased proliferation an/or decrease apoptosis. A slight increase in spontaneous proliferation of freshly isolated DKO splenocytes and LNC and a modest increase in the percentage of CD4, CD8 and B cells in S phase as evaluated by propidium iodide staining was observed. To measure spontaneous proliferation, DKO LN cells were plated at 2.5×10⁶ cells/ml in 96 well plates and 1 μCi/well of [³H]-thymidine was added at 6 hours and cells harvested 12 hours later.

More impressive, however, was the resistance of DKO T cells to antigen-induced cell death upon anti-CD3 stimulation. LN T cells from a tertiary stimulation were restimulated with 1 μg/ml of platebound anti-CD3 antibody for 20 hours, and TUNEL assay performed.

Given this data and the previous observation that induction of FasL after one hour was impaired in mice lacking NFATp, the induction of FasL was examined at later time points after TCR stimulation in DKO T cells. Unfractionated LNC were stimulated with 1 μg/ml of anti-CD3 antibody for 6 hours. RNA blots were hybridized with a FasL-specific probe [Takahashi, T. et al., Cell 76:969 (1994)]. An actin probe was used to verify equal RNA loading and a TCRα probe used to control for differences in T cell numbers.

Northern blot analysis revealed nearly complete absence of FasL transcripts in DKO T cells after 6 hours stimulation with anti-CD3. We conclude that the massive splenomegaly and lymphadenopathy observed in DKO mice is due at least in part to compromised FasL expression and defective apoptosis over time. However, there are clearly substantial differences between the phenotypes of the FasL deficient gld strain and the NFAT DKO strain, as discussed below.

These data and that of Koretsky and colleagues [Latinis, K. M. et al., J. Immunol 158:4602 (1997)] demonstrate that NFAT proteins regulate the FasL gene in vivo. However, the NFAT DKO phenotype cannot be solely explained on the basis of impaired FasL expression as evidenced by comparison with the phenotype of gld mice. NFAT DKO mice have rapid onset (by 7 weeks) of lymphadenopathy comprised of SP T cells and B cells, selectively elevated levels of Th2-type cytokines and the corresponding isotypes IgG1 and IgE, and no evidence of autoimmune disease although they do have anti-nuclear antibodies. Gld animals have a slower onset of lymphadenopathy (3-5 months) secondary to expansion of a DN B220+T cell subpopulation not present in NFAT DKO mice, hypergammaglobulinemia with especially elevated expression of the IgG2a isotype, no elevation of Th2-type cytokines and manifest autoimmunity with immune complex glomulonephritis [Takahashi, T. et al., Cell 76:969 (1994); Cohen, P. L. et al., Annu. Rev. Immunol. 9:243 (1991); Watanabe-Fukunaga, R. et al., Nature 356:314 (1992)].

### EXAMPLE 3

### NFATp/NFAT4 Doubly-Deficient Mice Exhibit Markedly Increased Th2 Cytokine Production

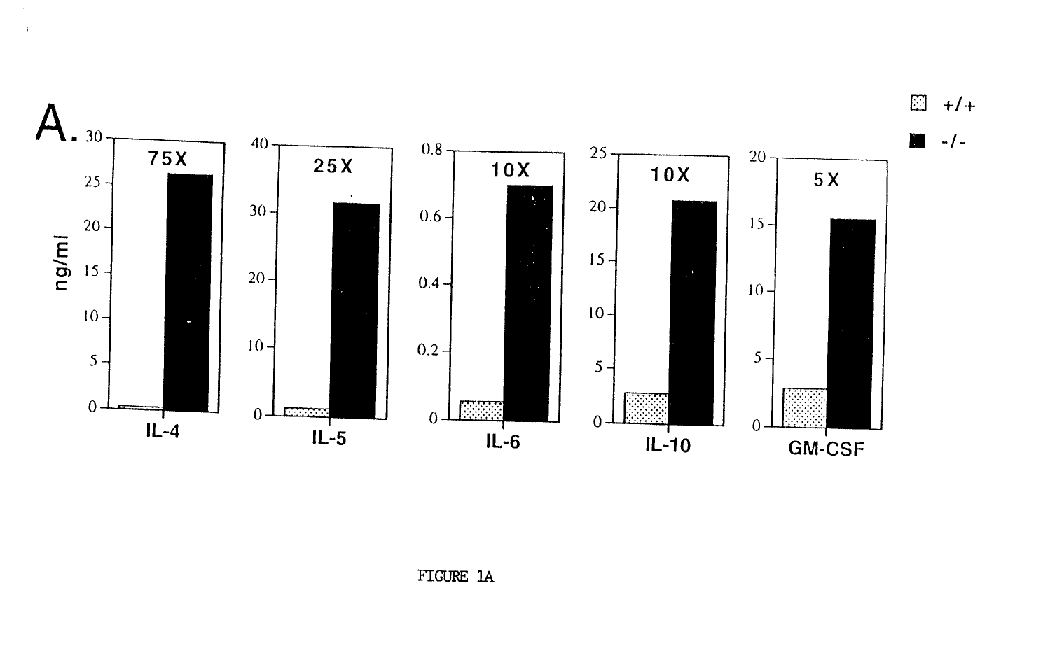

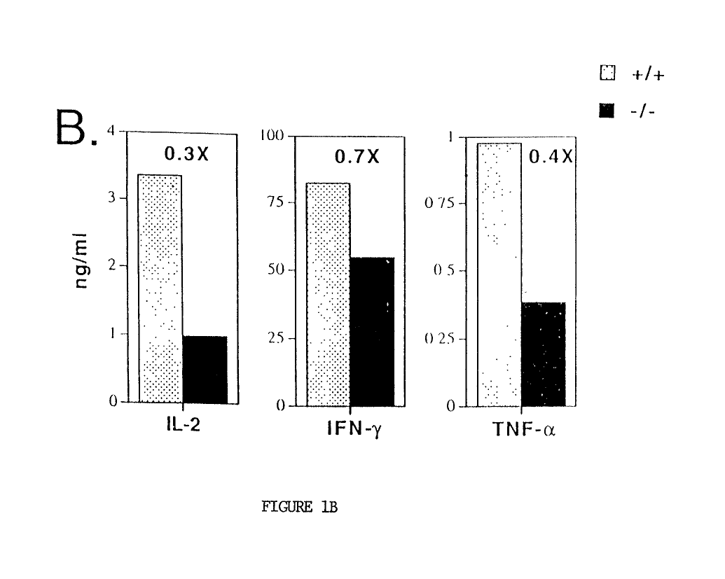

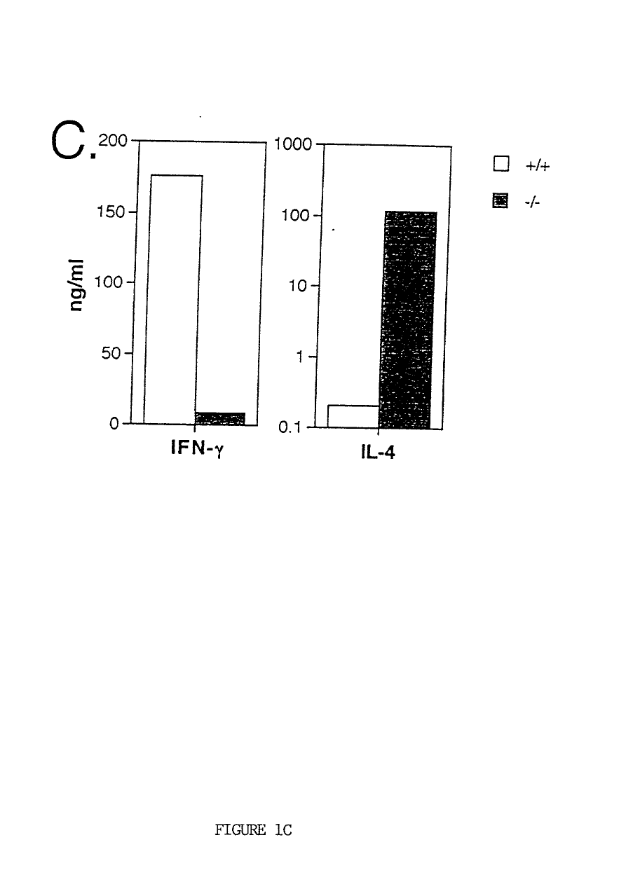

The presence of blepharitis, interstitial pneumonitis, increased mast cell numbers and granulomas in spleen and LN of NFAT DKO mice suggested overproduction of Th2-type cytokines in these animals. Indeed, a dramatic increase in Th2 cytokine production in response to anti-CD3 stimulation of DKO spleen and LN cells was observed. To examine cytokine production, freshly isolated splenocytes from wild-type or DKO mice were cultured at 2×10⁶ cells/ml with 1 ug/ml of plate-bound anti-CD3 for 48 hours. Cytokines (IL-2, IL-4, IL-5, IL-6, IL-10, GM-CSF, IFN-γ, TNFα) were measured by ELISA in supernatants taken at 24 hours for IL-2 and 48 hours for all others. For secondary stimulation of spleen cells from DKO mice, cytokines were measured as above at 48 hours. The results of this cytokine production analysis are summarized in the bar graphs of FIGS. 1A, 1B and 1C.

The amount of IL-4 produced by unfractionated DKO spleen cells in a primary response was approximately 75 fold greater than wild type and this increased to 600 fold in a secondary response. The levels of other Th2-type cytokines, IL-5, IL-6 and IL-10, were also very high. In contrast, levels of the Th1-type cytokines, IFN-γ, IL-2 and TNFα, were modestly to significantly decreased. Levels of GM-CSF, a cytokine produced by both Th I and Th2 cells were elevated, and together with IL-4 likely account for the formation of granulomas and infiltration of mast cells observed [Wynn, T. A. et al., Curr. Opin. Immunol. 7:505 (1995)].

This overproduction of IL-4 resulted in a massive increase in the levels of the IL-4 dependent isotypes IgG1 (2 to 3 logs) and IgE (3 to 4 logs) in the sera of unimmunized mice, as summarized below in Table 1. Serum immunoglobulin levels were determined by isotype-specific ELISA in 12-14 week-old wildtype (WT) or doubly-deficient (DKO) mice and are shown in μg/ml.

TABLE 1
Serum Immunoglobulin Levels from Wild-Type and
NFATp × NFAT4-Deficient (DKO) Mice

| | Age | | | | | | | |
|---|---|---|---|---|---|---|---|---|
| | (wks) | IgM | IgG1 | IgG2a | IgG2b | IgG3 | IgA | IgE |
| WT1 | 12 | 293 | 192 | 1193 | 109 | 340 | 125 | 0.2 |
| WT2 | 13 | 223 | 45 | 274 | 19 | 195 | 26 | 0.2 |
| WT3 | 14 | 230 | 22 | 201 | 10 | 180 | 26 | 0.1 |
| DKO1 | 12 | 1110 | 29,856 | 1233 | 139 | 386 | 118 | 755 |
| DKO2 | 13 | 1238 | 44,769 | 2441 | 243 | 1060 | 161 | 1,063 |
| DKO3 | 14 | 927 | 26,472 | 2920 | 799 | 184 | 182 | 828 |

The extraordinarily large amounts of IgE and IgG1 produced far exceed those present in single NFATp deficient mice [Hodge, M. R. et al, Immunity 4:1 (1996); Xanthoudakis, S. et al., Science 272:892 (1996)] or in mice that overexpress the IL-4 gene itself [Tepper, R. I. et al., Cell 62:457 (1990)]. In contrast to other mouse strains with lymphoproliferative disorders (CTLA4 deficient, lpr and gld strains), the hypergammaglobulinemia was very isotype specific as levels of IgG2a and 2b were only minimally increased. This is consistent with the nearly normal amounts of IFN-γ cytokines observed in the NFAT DKO.

The NFAT DKO phenotype can also not be solely attributed to elevated levels of Th2 cytokines, in particular IL-4. IL-4 overexpressor transgenics do have increased levels of IgE and allergic blepharitis [Tepper, R. I. et al., Cell 62:457 (1990)], but they are actually lymphopenic. Similar to the NFAT DKO mice, IL-2 receptor-β-deficient mice have high levels of immunoglobulins IgG1 and IgE but unlike them, also have autoimmune manifestations, infiltrative granulocytopoiesis, and further, lack lymphadenopathy [Suzuki, H. et al., Science 268:1472 (1995)].

Equivalents

Those skilled in the art will recognize, or be able to ascertain using no more than routine experimentation, many equivalents to the specific embodiments of the invention described herein. Such equivalents are intended to be encompassed by the following claims.

We claim:

1. A mouse comprising in its genome a first exogenous DNA molecule that functionally disrupts a NFATp gene of said mouse and a second exogenous DNA molecule that functionally disrupts a NFAT4 gene of said mouse, wherein said mouse exhibits a phenotype characterized by increased Th2 cytokine production, relative to a wildtype mouse.

2. The mouse of claim 1, wherein the phenotype of said mouse is further characterized by:
(a) blepharatis;
(b) interstitial pneumonitis;
(c) splenomegaly and lymphadenopathy; and
(d) increased levels of serum IgG1 and IgE, relative to a wildytype mouse.

3. A method of identifying a compound that regulates Th2 cell activity comprising:
a) contacting lymphoid cells deficient in NFATp and NFAT4 with a test compound; and
b) determining the effect of the test compound on an indicator of Th2 cell activity of the lymphoid cells, the test compound being identified as a modulator of Th2 cell activity based on the ability of the test compound to modulate an indicator of Th2 cell activity of the lymphoid cells deficient in NFATp and NFAT4.

4. The method of claim 3, wherein the lymphoid cells deficient in NFATp and NFAT4 are in a mouse that is deficient in NFATp and NFAT4 and the lymphoid cells are contacted with the test compound by administering the test compound to the mouse.

5. The method of claim 3, wherein the lymphoid cells deficient in NFATp and NFAT4 are isolated from a mouse deficient in NFATp and NFAT4 and the lymphoid cells are contacted with the test compound by culturing the test compound with the isolated lymphoid cells deficient in NFATp and NFAT4.

6. The method of claim 3, wherein the compound inhibits Th2 cytokine production.

7. A method of identifying a compound that regulates Th2 cell activity, comprising
a) providing at least one indicator composition comprising NFATp protein and NFAT4 protein
b) contacting the at least one indicator composition with each member of a library of test compounds;
c) selecting from the library of test compounds a compound of interest that modulates the activity of NFATp protein and NFAT4 protein; and
d) determining the effect of the compound of interest on Th2 cell activity to thereby identify a compound that regulates Th2 cell activity.

8. The method of claim 7, wherein the at least one indicator composition is at least one cell that expresses NFATp protein and NFAT4 protein.

9. The method of claim 8, wherein the at least one cell has been engineered to express NFATp protein and NFAT4 protein by introducing into the at least one cell at least one expression vector encoding the NFATp protein and the NFAT4 protein.

10. The method of claim 7, wherein the indicator composition is a cell free composition.

11. The method of claim 8, wherein the indicator composition is at least one cell that expresses an NFATp protein, an NFAT4 protein and at least one target molecule, and the ability of the test compound to modulate the interaction of the NFATp protein and the NFAT4 protein with the at least one target molecule is monitored.

12. The method of claim 8, wherein the indicator composition comprises at least one indicator cell, wherein the at least one indicator cell comprises an NFATp protein, an NFAT4 protein and at least one reporter gene responsive to the NFATp protein or the NFAT4 protein.

13. The method of claim 12, wherein the at least one indicator cell contains: at least one recombinant expression vector encoding the NFATp protein and the NFAT4 protein; and at leat one vector comprising at least one NFATp- or NFAT4-responsive regulatory element operatively linked to at least one reporter gene; and said method comprises:
a) contacting the at least one indicator cell with a test compound;
b) determining the level of expression of the at least one reporter gene in the at least one indicator cell in the presence of the test compound; and
c) comparing the level of expression of the at least one reporter gene in the at least one indicator cell in the presence of the test compound with the level of expression of the at least one reporter gene in the at least one indicator cell in the absence of the test compound to thereby select a compound of interest that modulates the activity of NFATp protein or NFAT4 protein.

14. A method for modulating Th2 cell activity, comprising contacting lymphoid cells with a modulator of NFATp and NFAT4 activity such that Th2 cell activity within the lymphoid cells is modulated.

15. The method of claim 14, wherein the modulator inhibits NFATp and NFAT4 activity.

16. The method of claim 15, wherein the modulator is at least one antisense oligonucleotide.

17. The method of claim 15, wherein the modulator is at least one intracellular antibody.

18. The method of claim 15, wherein the modulator is at least one peptidic compound derived from the calcineurin-interacting region of NFATp or NFAT4.

19. The method of claim 18, wherein the modulator comprises the amino acid sequence of SEQ ID NO: 1.

20. The method of claim 18, wherein the peptide comprises the amino acid sequence of SEQ ID NO: 2 or SEQ ID NO: 3.

21. The method of claim 14, wherein the modulator stimulates NFATp and NFAT4 activity.

22. The method of claim 21, wherein the modulator is at least one expression vector encoding NFATp and NFAT4.

23. The method of claim 14, wherein the lymphoid cells are contacted with the modulator by culturing the cells in vitro with the modulator.

24. The method of claim 23, wherein the lymphoid cells are contacted with a modulator that inhibits NFATp and NFAT4 activity such that Th2 cell activity is stimulated, the method further comprising administering the lymphoid cells having increased Th2 cell activity to a subject.

25. The method of claim 14, wherein the modulator is contacted with the lymphoid cells by administering the modulator to a subject.

26. A method of diagnosing a subject for a disorder associated with aberrant Th2 cell activity comprising:
(a) detecting expression of NFATp and NFAT4 in lymphoid cells of a subject suspected of having a disorder associated with aberrant Th2 cell activity;
(b) comparing expression of NFATp and NFAT4 in lymphoid cells of said subject to a control that is not associated with aberrant Th2 cell activity; and
(c) diagnosing the subject for a disorder based a change in expression of NFATp or NFAT4 in lymphoid cells of the subject as compared to the control.

27. The method of claim 26, wherein expression of NFATp or NFAT4 in lymphoid cells of the subject is elevated relative to the control.

28. The method of claim 26, wherein expression of NFATp or NFAT4 in lymphoid cells of the subject is reduced relative to the control.

29. The method of claim 26, wherein a mutant form of NFATp or NFAT4 is expressed in lymphoid cells of the subject compared to the control.

30. The method of claim 26, wherein the expression of NFATp or NFAT4 is detected in vivo in the subject.

31. The method of claim 26, wherein the expression of NFATp or NFAT4 is detected in lymphoid cells isolated from the subject
